SEQUENCE LISTING (57 sequences)

SEQ ID NO: 1 - protein, 25 aa, synthetic construct
  source 1..25: note = HFWR1
EVQLQQSGAELMKPGASVKISCKAS

SEQ ID NO: 2 - protein, 25 aa, synthetic construct
  source 1..25: note = HFWR1
EVQLVESGGGLVQPGGSLKLSCAAS

SEQ ID NO: 3 - protein, 25 aa, synthetic construct [sequence omitted]

SEQ ID NO: 4 - protein, 7 aa, synthetic construct
  source 1..7: note = HCDR1
GYTFSDY

SEQ ID NO: 5 - protein, 7 aa, synthetic construct
  source 1..7: note = HCDR1
GFTFNTY

SEQ ID NO: 6 - protein, 7 aa, synthetic construct
  source 1..7: note = HCDR1
GYTFTSY

SEQ ID NO: 7 - protein, 19 aa, synthetic construct
  source 1..19: note = HFWR2
WIEWVKQRPGHGLEWIGEI

SEQ ID NO: 8 - protein, 19 aa, synthetic construct
  source 1..19: note = HFWR2
AMNWVRQASGKGLEWVGRI

SEQ ID NO: 9 - protein, 19 aa, synthetic construct
  source 1..19: note = HFWR2
NMHWVRQAPGQGLEWMGVI

SEQ ID NO: 10 - protein, 6 aa, synthetic construct
  source 1..6: note = HCDR2
LPGSGS

SEQ ID NO: 11 - protein, 8 aa, synthetic construct
  source 1..8: note = HCDR2
RSKYNNYA

SEQ ID NO: 12 - protein, 6 aa, synthetic construct
  source 1..6: note = HCDR2
YSGNGD

SEQ ID NO: 13 - protein, 41 aa, synthetic construct
  source 1..41: note = HFWR3
TNYHERFKGKATFTADTSSSTAYMQLNSLTSEDSGVYYCLH

SEQ ID NO: 14 - protein, 41 aa, synthetic construct
  source 1..41: note = HFWR3
TYYADSVKDRFTISRDDSKSTLYLQMNSLKTEDTAVYYCVR

SEQ ID NO: 15 - protein, 41 aa, synthetic construct
  source 1..41: note = HFWR3
TSYNQKFKGRVTITADKSTSTAYMELSSLRSEDTAVYYCAR

SEQ ID NO: 16 - protein, 7 aa, synthetic construct
  source 1..7: note = HCDR3
GNYDFDG

SEQ ID NO: 17 - protein, 14 aa, synthetic construct
  source 1..14: note = HCDR3
HGNFGNSYVSWFAY

SEQ ID NO: 18 - protein, 8 aa, synthetic construct
  source 1..8: note = HCDR3
ERDTRFGN

SEQ ID NO: 19 - protein, 11 aa, synthetic construct
  source 1..11: note = HFWR4
WGQGTTLTVSS

SEQ ID NO: 20 - protein, 11 aa, synthetic construct
  source 1..11: note = HFWR4
WGQGTLVTVSS

SEQ ID NO: 21 - protein, 23 aa, synthetic construct
  source 1..23: note = LFWR1
DIVLTQSPAIMSASPGEKVTMTC

SEQ ID NO: 22 - protein, 22 aa, synthetic construct
  source 1..22: note = LFWR1
QAVVTQEPSLTVSPGGTVTLTC

SEQ ID NO: 23 - protein, 23 aa, synthetic construct
  source 1..23: note = LFWR1
DIVMTQSPDSLAVSLGERATINC

SEQ ID NO: 24 - protein, 10 aa, synthetic construct
  source 1..10: note = LCDR1
SASSSVNYMY

SEQ ID NO: 25 - protein, 14 aa, synthetic construct
  source 1..14: note = LCDR1
RSSTGAVTTSNYAN

SEQ ID NO: 26 - protein, 15 aa, synthetic construct
  source 1..15: note = LCDR1
RASESVDIYGNSFMH

SEQ ID NO: 27 - protein, 15 aa, synthetic construct
  source 1..15: note = LFWR2
WYQQKSGTSPKRWIY

SEQ ID NO: 28 - protein, 15 aa, synthetic construct
  source 1..15: note = LFWR2
WVQQKPGQAPRGLIG

SEQ ID NO: 29 - protein, 15 aa, synthetic construct
  source 1..15: note = LFWR2
WYQQKPGQPPKLLIY

SEQ ID NO: 30 - protein, 7 aa, synthetic construct
  source 1..7: note = LCDR2
DTSKLAS

SEQ ID NO: 31 - protein, 7 aa, synthetic construct
  source 1..7: note = LCDR2
GTNKRAP

SEQ ID NO: 32 - protein, 7 aa, synthetic construct
  source 1..7: note = LCDR2
LASNLES

SEQ ID NO: 33 - protein, 32 aa, synthetic construct
  source 1..32: note = LFWR3
GVPVRFSGSGSGTSYSLTISSMETEDAAEYYC

SEQ ID NO: 34 - protein, 32 aa, synthetic construct
  source 1..32: note = LFWR3
WTPARFSGSLLGDKAALTLLGAQPEDEAEYFC

SEQ ID NO: 35 - protein, 32 aa, synthetic construct
  source 1..32: note = LFWR3
GVPDRFSGSGSGTDFTLTISSLQAEDVAVYYC

SEQ ID NO: 36 - protein, 8 aa, synthetic construct
  source 1..8: note = LCDR3
QQWGRNPT

SEQ ID NO: 37 - protein, 9 aa, synthetic construct
  source 1..9: note = LCDR3
ALWYSNLWV

SEQ ID NO: 38 - protein, 9 aa, synthetic construct
  source 1..9: note = LCDR3
QQNNEDPYT

SEQ ID NO: 39 - protein, 10 aa, synthetic construct
  source 1..10: note = LFWR4
FGGGTKLEIK

SEQ ID NO: 40 - protein, 10 aa, synthetic construct
  source 1..10: note = LFWR4
FGGGTKLTVL

SEQ ID NO: 41 - protein, 10 aa, synthetic construct
  source 1..10: note = LFWR4
FGGGTKVEIK

SEQ ID NO: 42 - protein, 116 aa, synthetic construct
  source 1..116: note = VH
EVQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSS

SEQ ID NO: 43 - protein, 125 aa, synthetic construct
  source 1..125: note = VH
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSS

SEQ ID NO: 44 - protein, 117 aa, synthetic construct
  source 1..117: note = VH
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 45 - protein, 105 aa, synthetic construct
  source 1..105: note = VL
DIVLTQSPAIMSASPGEKVTMTCSASSSVNYMYWYQQKSGTSPKRWIYDTSKLASGVPVRFSGSGSGTSYSLTISSMETEDAAEYYCQQWGRNPTFGGGTKLEIK

SEQ ID NO: 46 - protein, 109 aa, synthetic construct
  source 1..109: note = VL
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVL

SEQ ID NO: 47 - protein, 111 aa, synthetic construct
  source 1..111: note = VL
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 48 - protein, 446 aa, synthetic construct
  source 1..446: note = HC
EVQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 455 aa, synthetic construct
  source 1..455: note = HC
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 447 aa, synthetic construct
  source 1..447: note = HC
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 212 aa, synthetic construct
  source 1..212: note = LC
DIVLTQSPAIMSASPGEKVTMTCSASSSVNYMYWYQQKSGTSPKRWIYDTSKLASGVPVRFSGSGSGTSYSLTISSMETEDAAEYYCQQWGRNPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 215 aa, synthetic construct
  source 1..215: note = LC
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 53 - protein, 218 aa, synthetic construct
  source 1..218: note = polypeptide chain
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 219 aa, synthetic construct
  source 1..219: note = polypeptide chain
EVQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 440 aa, synthetic construct
  source 1..440: note = polypeptide chain
DIVLTQSPAIMSASPGEKVTMTCSASSSVNYMYWYQQKSGTSPKRWIYDTSKLASGVPVRFSGSGSGTSYSLTISSMETEDAAEYYCQQWGRNPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 220 aa, synthetic construct
  source 1..220: note = polypeptide chain
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 446 aa, synthetic construct
  source 1..446: note = polypeptide chain
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2026/0217820 A1
(43) Pub. Date: Jul. 30, 2026

(54) BISPECIFIC ANTIBODIES TARGETING CD117 AND CD3

(71) Applicants: ERASMUS UNIVERSITY MEDICAL CENTER ROTTERDAM, Rotterdam (NL); HARBOUR BIOMED (SHANGHAI) CO., LTD., Shanghai (CN)

(72) Inventors: Sj ERKELAND, Rotterdam (NL); Pd KATSIKIS, Rotterdam (NL); Yun HE, Shanghai (CN); Yuandong WANG, Shanghai (CN)

(21) Appl. No.: 19/141,697

(22) PCT Filed: Dec. 20, 2023
(86) PCT No.: PCT/CN2023/140352

§ 371 Date: Jun. 20, 2025

(30) Foreign Application Priority Data
Dec. 21, 2022 (WO) PCT/CN2022/140683

(51) Int. Cl. C07K 16/28; A61K 39/00; A61K 47/68; A61P 35/02

(52) U.S. Cl. CPC C07K 16/2803; A61K 47/6801; A61K 47/6849; A61P 35/02; C07K 16/2809; A61K 2039/505; C07K 2317/31; C07K 2317/33; C07K 2317/73; C07K 2317/732; C07K 2317/92

(57) ABSTRACT

Provided herein are bispecific antibodies comprising a first antigen binding region and a second antigen binding region, wherein the first antigen binding region binds to CD117 and comprises a first heavy chain variable region and a first light chain variable region, and the second binding region binds to CD3 and comprises a second heavy chain variable region and a second light chain variable region. Also provided are compositions comprising the bispecific antibodies as well as uses of the bispecific antibodies and compositions.

### CROSS-REFERENCE TO RELATED APPLICATION

This application claims priority to and the benefit of International Application No. PCT/CN2022/140683 filed on Dec. 21, 2022, which is incorporated herein by reference in its entirety for all purposes.

### FIELD OF THE INVENTION

The invention relates to bispecific antibodies binding to CD117 and CD3 and antigen-binding fragment thereof, as well as use of the bispecific antibodies.

### BACKGROUND OF THE INVENTION

Hematopoietic stem cell transplantation has proven to be effective for the treatment of hematopoietic disorders. However, current therapies of hematopoietic stem cell transplantation are often associated with significant toxicity due to use of radiation or chemotherapy that put the patient at high risk of life-threatening infections and toxicity. Severe infections frequently present in the immediate post-hematopoietic stem cell transplantation period, while complications caused by chemotoxicity and irradiation damage affect multiple organs at the long term. Antibodies that rapidly and transiently deplete hematopoietic stem cells to create the desired bone marrow space allow for sufficient hematopoietic stem cell engraftment. Highly effective hematopoietic stem cell depleting antibodies that have a short half-life in the body would be ideal for rapid and efficient treatment of patients.

Antibody-mediated conditioning of the host bone marrow prior to hematopoietic stem cell transplantation is promising but not optimal yet. Among the targets for hematopoietic stem cell depletion, the CD117 receptor is a potential target expressed on all hematopoietic stem cells. Czechowicz and colleagues showed that the administration of the anti-CD117 antibody ACK2 blocked CD117 function and thereby led to a transient loss of>98% of endogenous hematopoietic stem cells in mice (Czechowicz A, et al., Science. 2007; 318 (5854): 1296-1299). A different study of Czechowicz showed that an anti-CD117-antibody-drug-conjugates (CD117-ADC) to Saporin leads to a >99% depletion of hematopoietic stem cells in mice (Czechowicz A, et al. Nat.Commun. 2019; 10 (1): 617).

In human, the available antibodies are less effective. An anti-human CD117 antibody, the so-called SR-1, inhibits hematopoietic stem cells in vitro. Humanized SR-1 (AMG191) is capable of depleting hematopoietic stem cells by inhibiting CD117 signaling (WO 2019/113437 A1; Pang W. W. et al., Blood. 2019; 133 (19): 2069-2078). In non-human primates, the circulation time of AMG191 is dependent on the dose, with a T½ of 1.88 (0.1 mg/kg) to 4.3 days (25 mg/kg). In non-human primates and human bone marrow CD34+ xenografted immune-deficient mice, AMG191 treatment depleted endogenous hematopoietic stem cells and conditioned the bone marrow for hematopoietic stem cell engraftment (Kwon H. S. et al., Blood. 2019; 133 (19): 2104-2108). However, the first results of an ongoing clinical trial (Trial: NCT02963064) using CD117-targeting with full length AMG191 antibody for conditioning in patients with severe combined immunodeficiency (SCID), report low percentages of chimerism (3-10%) after allogeneic hematopoietic stem cell transplantation (Agarwal R., et al., Biology of Blood and Marrow Transplantation. 2019; 25 (3): S92). In this study, hematopoietic stem cell transplantation was only performed when antibody plasma levels reached <100 ng/mL, which takes up to 20 days post-treatment. Due to the long time-period between the injection of antibodies and reaching the level suitable for hematopoietic stem cell transplantation, the simultaneous recovery of the endogenous hematopoietic stem cell pool is a potential problem of this strategy.

There is a need for rapid and low-risk hematopoietic stem cell depletion for hematopoietic stem cell transplantation approach. Agents with a short half-life and high hematopoietic stem cell depletion capacity would be preferable for safe bone marrow conditioning and rapid hematopoietic stem cell transplantation.

### SUMMARY OF THE INVENTION

In a first aspect, the invention provides a bispeicific antibody or an antigen binding fragment thereof, comprising a first antigen binding region and a second antigen binding region, wherein the first antigen binding region binds to CD117 and comprises a first heavy chain variable region (VH_A) and a first light chain variable region (VL_A), and the second binding region binds to CD3 and comprises a second heavy chain variable region (VH_B) and a second light chain variable region (VL_B), wherein:
the VH_A comprises a HCDR1, a HCDR2 and a HCDR3 of a VH comprising the amino acid sequence of SEQ ID NO: 44;
the VL_A comprises a LCDR1, a LCDR2 and a LCDR3 of a VL comprising the amino acid sequence of SEQ ID NO: 47;
the VH_B comprises a HCDR1, a HCDR2 and a HCDR3 of a VH comprising the amino acid sequence of SEQ ID NO: 43; and
the VL_B comprises a LCDR1, a LCDR2 and a LCDR3 of a VL comprising the amino acid sequence of SEQ ID NO: 46.

In some embodiments of the bispecific antibody or antigen binding fragment thereof, the VH_A comprises a HCDR1, a HCDR2 and a HCDR3 having the amino acid sequences of SEQ ID NOs: 6, 12 and 18 respectively;
the VL_A comprises a LCDR1, a LCDR2 and a LCDR3 having the amino acid sequences of SEQ ID NOs: 26, 32 and 38 respectively;
the VH_B comprises a HCDR1, a HCDR2 and a HCDR3 having the amino acid sequences of SEQ ID NOs: 5, 11 and 17 respectively; and
the VL_B comprises a LCDR1, a LCDR2 and a LCDR3 having the amino acid sequences of SEQ ID NOs: 25, 31 and 37 respectively.

In some embodiments of the bispecific antibody or antigen binding fragment thereof, the VH_A comprises an amino acid sequence having at least 80%, at least 85%, at least 90%, at least 95%, at least 98%, at least 99%, or 100% sequence identity to SEQ ID NO: 44; the VL_A comprises an amino acid sequence having at least 80%, at least 85%, at least 90%, at least 95%, at least 98%, at least 99%, or 100% sequence identity to SEQ ID NO: 47; the VH_B comprises an amino acid sequence having at least 80%, at least 85%, at least 90%, at least 95%, at least 98%, at least 99%, or 100% sequence identity to SEQ ID NO: 43; and the VL_B comprises an amino acid sequence having at least 80%, at least 85%, at least 90%, at least 95%, at least 98%, at least 99%, or 100% sequence identity to SEQ ID NO: 46.

In some embodiments of the bispecific antibody or antigen binding fragment thereof,
the VH_A comprises an amino acid sequence as set forth in SEQ ID NO: 44;
the VL_A comprises an amino acid sequence as set forth in SEQ ID NO: 47;
the VH_B comprises an amino acid sequence as set forth in SEQ ID NO: 43; and
the VL_B comprises an amino acid sequence as set forth in SEQ ID NO: 46.

In some other embodiments of the bispecific antibody or antigen binding fragment thereof, the antibody comprises three polypeptide chains, wherein:
the first polypeptide chain comprises from the N terminal to C terminal: VH_A-CH1; the second polypeptide chain comprises from the N terminal to C terminal: VL_A-CL-L-VH_B-CH1; and the third polypeptide chain comprises from the N terminal to C terminal: VL_B-CL; or
the first polypeptide chain comprises from the N terminal to C terminal: VH_B-CH1; the second polypeptide chain comprises from the N terminal to C terminal: VL_B-CL-L-VH_A-CH1; and the third polypeptide chain comprises from the N terminal to C terminal: VL_A-CL,
wherein CH1 represents the first domain of constant region of an immunoglobulin heavy chain; CL each represents constant region of an immunoglobulin light chain; and L is absent or represent an optional linker.

In some embodiments, the CL in the second polypeptide chain and the CL in the third polypeptide chain each independently comprises a constant region derived from λ light chain or κ light chain.

In some embodiments, the CL in the second polypeptide chain and the CL in the third polypeptide chain are derived from different types of immunoglobulin light chains

In some embodiments of the bispecific antibody or antigen binding fragment thereof, the CL in the second polypeptide chain comprises a light chain constant region derived from λ light chain (e.g. human λ light chain), and the CL in the third polypeptide chain comprises a light chain constant region derived from κ light chain (e.g. human κ light chain).

In some other embodiments of the bispecific antibody or antigen binding fragment thereof, the CL in the second polypeptide chain comprises a light chain constant region derived from κ light chain (e.g. human κ light chain), and the CL in the third polypeptide chain comprises a light chain constant region derived from 2 light chain (e.g. human λ light chain).

In some embodiments of the bispecific antibody or antigen binding fragment thereof, the VL_A comprises a light chain variable region derived from the same type of immunoglobulin light chain as the CL in the same polypeptide chain, and/or the VL_B comprises a light chain variable region derived from the same type of immunoglobulin light chain as the CL in the same polypeptide chain.

In some embodiments of the bispecific antibody or antigen binding fragment thereof, each of the CH1 independently comprises a CH1 derived from immunoglobulin isotype IgG (e.g. human IgG), optionally each of the CH1 independently comprises a CH1 derived from IgG subtype selected from the group consisting of IgG1, IgG2 and IgG4 (e.g. human IgG1, IgG2 and IgG4).

In some embodiments of the bispecific antibody or antigen binding fragment thereof, the linker comprises an amino acid sequence of (G4S) n, wherein n is an integer selected from 1-5, e.g. 1, 2, 3, 4 or 5.

In some embodiments of the bispecific antibody or antigen binding fragment thereof,
the first polypeptide chain comprises an amino acid sequence having at least 80%, at least 85%, at least 90%, at least 95%, at least 98%, at least 99%, or 100% sequence identity to SEQ ID NO: 57; the second polypeptide chain comprises an amino acid sequence having at least 80%, at least 85%,
at least 90%, at least 95%, at least 98%, at least 99%, or 100% sequence identity to SEQ ID NO: 56; and
the third polypeptide chain comprises an amino acid sequence having at least 80%, at least 85%, at least 90%, at least 95%, at least 98%, at least 99%, or 100% sequence identity to SEQ ID NO: 52.

In some embodiments of the bispecific antibody or antigen binding fragment thereof,
the first polypeptide chain comprises an amino acid sequence as set forth in SEQ ID NO: 57;
the second polypeptide chain comprises an amino acid sequence as set forth in SEQ ID NO: 56; and
the third polypeptide chain comprises an amino acid sequence as set forth in SEQ ID NO: 52.

In some embodiments of the bispecific antibody or antigen binding fragment thereof, the antibody does not comprise an immunoglobulin Fc region.

In a second aspect, the invention provides a nucleic acid (e.g. an isolated nucleic acid) encoding the bispecific antibody or antigen binding fragment thereof of the first aspect of the invention.

In a third aspect, the invention provides a vector comprising the nucleic acid of the second aspect of the invention. In some preferred embodiments, the vector is an expression vector.

In a fourth aspect, the invention provides a host cell which comprises the nucleic acid of the second aspect of the invention or the vector of the third aspect of the invention.

In a fifth aspect, the invention provdes a method of preparing the bispecific antibody or antigen binding fragment thereof of the first aspect of the invention, wherein the method comprises
a) culturing the host cell of the fourth aspect of the invention under a condition suitable for the production of the bispecific antibody or antigen binding fragment thereof; and
b) obtaining the bispecific antibody or antigen binding fragment thereof from the culture.

In a sixth aspect, the invention provides a antibody drug conjugate (ADC) comprising the bispecific antibody or antigen binding fragment thereof of the first aspect of the invention, and a cytotoxic moiety conjugated to the bispecific antibody or antigen binding fragment thereof.

In a seventh aspect, the invention provides a composition comprising the bispecific antibody or antigen binding fragment thereof of the first aspect of the invention. In some embodiments, the composition is a pharmaceutical composition and further comprises a pharmaceutically acceptable carrier and/or excipient.

In a eighth aspect, the invention provides a method of depleting hematopoietic stem cells in a subject in need thereof, comprising administrating the bispecific antibody or antigen binding fragment thereof of the first aspect of the invention, the nucleic acid of the second aspect of the invention, the vector of the third aspect of the invention, the ADC of the sixth aspect of the invention, or the composition of the seventh aspect of the invention to the subject.

In a ninth aspect, the invention provdes a method of treating a hematopoietic disorder in a subject in need thereof, comprising:
a) administrating the bispecific antibody or antigen binding fragment thereof of the first aspect of the invention, the ADC of the sixth aspect of the invention, or the composition of the seventh aspect of the invention to the subject; and
b) transplanting hematopoietic stem cells to the subject.

In some embodiments of the method, the step b) is performed within 24 hours, within 48 hours, within 72 hours, within 5 days, within 7 days, within 2 weeks, within 3 weeks or within 1 month after step a).

In some embodiments, the hematopoietic disorder is seleted from the gourp consisting of a hemoglobinopathy, an immunodeficiency, a metabolic disorder, a hematological disorder (such as bone marrow failure syndromes, myeloid dysplastic syndromes, acute lymphoid and myeloid leukemia, and chronic lymphoid and myeloid leukemia), a hematopoietic cell proliferative disease, transplant rejection, an autoimmune disorder and an autoinflammatory disorder.

### DESCRIPTION OF THE DRAWINGS

FIG. 1: Schematic structure of PR004384 anti-CD117xCD3 Bispecific T cell engager

FIG. 2: PR004384 anti-CD117xCD3 binds specifically to CD117-positive hematopoietic cells and CD3-positive T cells
(A) FACS plots showing the binding of PR004384 anti-CD117xCD3 and control anti-HELxCD3 Bispecific T cell engager to CD117 expressed on TF-1 cells. RPMI-8226 and U937 cell lines were used as negative controls. (B) Affinity of anti-CD117xCD3 Bispecific T cell engager to TF-1 cells as determined by flow cytometry. (C) FACS plots showing the binding of PR004384 anti-CD117xCD3 and control anti-HELxCD3 Bispecific T cell engager to CD3 expressed on T cells and PBMCs. T cells and PBMCs pre-treated with anti-CD3 blocking antibodies were used as negative controls.

FIG. 3: PR004384 anti-CD117xCD3 promotes activation of T cells
(A) FACS plots showing proliferation of T cells under the indicated conditions. Gates show reduced CellTrace FarRed signals and the percentage of cells that proliferate. (B) FACS plots and quantification of the activation markers CD69 and CD25 expressed on CD4+ and CD8+ T cells when T cells and TF-1 cells were cultured together in the presence of indicated reagents. (C) Determination of the cytokine production of purified T cells when co-cultured with TF-1 cells in the presence of the indicated Bispecific T cell engager concentrations in vitro. Supernatants were collected after TDCC assay. Levels of cytokines in the supernatants are shown.

FIG. 4: PR004384 anti-CD117xCD3 efficiently depletes human CD117-positive hematopoietic cells in vitro
(A) Percentage of T-cell-mediated depletion of TF-1 cells (left) and hCD34+ cells (right) when treated with PR004384 anti-CD117xCD3 Bispecific T cell engager or control HELxCD3 Bispecific T cell engager in vitro. (B) Percentage of living TF-1 cells when treated with PR004384 anti-CD117xCD3 Bispecific T cell engager or control anti-HELxCD3 Bispecific T cell engager together with purified mouse T cells.

FIG. 5: Determination of the in vivo T½ in serum of anti-CD117xCD3 Bispecific T cell engager. Blood samples were taken at indicated time points and concentration of the Bispecific T cell engager in the serum was determined by ELISA.

FIG. 6: PR004384 anti-CD117xCD3 efficiently depletes human CD117-positive hematopoietic cells in vivo in humanized mice
(A) Treatment protocol of humanized mice. (B) Representative examples of FACS plots showing the percentage of human CD34+ CD117+ cells of total human CD45+ bone marrow cells in humanized mice after indicated treatment and at specified time points. (C) Graph showing percentage of CD34+ CD117+ cells of total human bone marrow cells in humanized mice after indicated treatment and at specified time points. (D) Graph showing total number of hCD45+ cells in the bone marrow of humanized mice after indicated treatment at 6 weeks post-treatment.

FIG. 7: PR004382 anti-CD117 antibody binds specifically to CD117 and TF-1 cells
(A) Results of ELISA-based binding assay of PR004382 anti-CD117 antibody and hIgG1 control to CD117 protein are shown. The EC50-value of PR004382 anti-CD117 antibody is depicted in the graph. (B) Results of TF-1 FACS-based binding assay of PR004382 anti-CD117 antibody and hIgG1 control are shown. The EC50-value of PR004382 anti-CD117 antibody is shown. (C) Binding and dissociation curves for the determination of the on-rate constant (K-on) and the dissociation constant (K-dis) to calculate the affinity (KD-value) of PR004382 anti-CD117 antibody to CD16a.

FIG. 8: PR004382 anti-CD117 antibody eliminates CD117-positive cells in in vitro killing assays
(A) Results of luminescence-based ADCC assay of PR004382 anti-CD117 antibody and hIgG1 control are shown. The EC50-value of PR004382 anti-CD117 antibody is depicted in the graph. (B) Percentages of NK-cell-mediated depletion of TF-1 cells when treated with different concentrations of PR004382 anti-CD117 antibody or hIgG1 controls antibody in vitro are shown. (C) Percentages of NK-cell-mediated depletion of human bone marrow CD34+ cells when treated with PR004382 ant-CD117 antibody or hIgG1 controls antibody (1 μg/mL) in vitro are shown (D) Determination of the in vivo T½ value in serum of PR004382. Blood samples were taken at indicated time points and concentration of the antibody in the serum was determined by ELISA.

FIG. 9: PR004384 anti-CD117xCD3 depletes primary human leukemia cells in vitro Purified leukemia cells from a patient with c-KIT-positive acute leukemia cells (T/myeloid mixed phenotype acute leukemia) were mixed with purified T cells and treated with PR004384 anti-CD117xCD3 or control anti-HELxCD3 at the indicated concentrations. The graph shows the percentage of patient leukemia cell death at 24 hours after indicated treatment.

FIG. 10: PR004384 anti-CD117xCD3 depletes human AML cells including leukemia initiating cells
(A) Treatment scheme of humanized mice transplanted with patient-derived xenograft (PDX) AML cells. (B) FACS plot showing the expression of hCD117 and Hcd33 on PDX AML cells. (C) Graph showing the number of human CD33- and CD117-positive PDX AML cells in the bone marrow of mice transplanted with PDX AML cells after indicated treatment. (D) Bone marrow cells of mice with indicated treatment were isolated and transplanted in secondary recipients. Graphs showing the number of AML cells in bone marrow (BM), spleen and the percentage of AML cells in the peripheral blood (PB) of secondary recipients 6 months post-transplantation.

FIG. 11: PR004384 anti-CD117xCD3 binds to Rhesus Macaque bone marrow HSCs
A) FACS plot showing the expression of CD117 and CD34 on bone marrow cells of Rhesus Macaque after CD34 enrichment. B) FACS plots showing binding of PR004384 anti-CD117xCD3 Bispecific T cell engager to Rhesus Macaque CD117-positive hematopoietic stem cells.

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland; Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

Unless indicated or defined otherwise, the term “comprise”, and variations such as “comprises” and “comprising”, should be understood to imply the inclusion of a stated elements or step or group of elements or steps but not the exclusion of any other element or step or group of elements or steps. The term “comprising” encompasses “including” as well as “consisting” e.g., a composition “comprising” X may consist exclusively of X or may include something additional e.g., X+Y.

The term “about” in relation to a numerical value x is optional and means, for example, x±10% or x±5%.

As used herein, the term “antibody” refers to an immunoglobulin molecule which has the ability to specifically bind to a specific antigen. An antibody often comprises a variable region and a constant region in each of a heavy chain and a light chain. The variable regions of the heavy and light chains of antibodies contain a binding domain that interacts with an antigen. The heavy chain constant region comprises three domains, e.g., CH1, CH2 and CH3. The light chain constant region comprises one domain, CL. The constant regions of antibodies may mediate the binding of the immunoglobulin to host tissues or factors, including various cells of the immune system (such as effector cells) and components of the complement system such as C1q, the first component in the classical pathway of complement activation. Accordingly, most antibodies have a heavy chain variable region (VH) and a light chain variable region (VL) that together form the portion of the antibody that binds to the antigen.

A “light chain variable region” (VL) or “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering system) in which corresponding residues between different heavy chains or between different light chains are assigned the same number. The present disclosure can use CDRs defined according to any of these numbering systems, although preferred embodiments use Chothia defined CDRs.

Based on the amino acid sequence of heavy chain constant regions of the antibody, a immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (λ) chain or a kappa (κ) chain, based on the amino acid sequence of the light chain.

The term “antibody” as used herein should be understood in its broadest meaning, and includes monoclonal antibodies (including full-length monoclonal antibodies), polyclonal antibodies, antibody fragments, and multi-specific antibodies containing at least two different antigen binding regions (e.g., bi-specific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

The term “bispecific antibody” is in the context of the present invention to be understood as an antibody having two different antigen-binding regions defined by different antibody sequences. This can be understood as different target binding but includes as well binding to different epitopes in one target. The term “bispecific antibody” as used herein should be understood in its broadest meaning, and includes full-length bispecific antibodies and antigen binding fragments thereof. The bispecific antibody may contain additional modifications, such as non-naturally occurring amino acids, mutations in Fc regions, and mutations in glycosylation sites. Bispecific antibodies also include post-translation modified antibodies, fusion proteins containing the antigenic determinants of the antibody, and immunoglobulin molecules containing any other modifications to antigen recognition sites, as long as these antibodies exhibit desired biological activity.

As used herein, the term “antigen binding fragment” of an antibody refers to one or more fragments of an antibody that retain the ability to specifically bind to an antigen. It has been shown that the antigen binding function of an antibody can be performed by fragments of a full-length antibody.

Examples of antigen binding fragments encompassed within the term “antigen binding portion” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fab′ fragment, which is essentially an Fab with part of the hinge region; (iv) a Fd fragment consisting of the VH and CH1 domains; (v) a Fd′ fragment having VH and CH1 domains and one or more cysteine residues at the C-terminus of the CH1 domain; (vi) a Fv fragment consisting of the VL and VH domains of a single arm of an antibody, (vii) a dAb fragment, which consists of a VH domain; (viii) an isolated complementarity determining region (CDR); and (ix) a nanobody, a heavy chain variable region containing a single variable domain and two constant domains. Furthermore, although the two domains of the Fv fragment, V Land VH are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. Furthermore, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity.

These antigen binding fragments can be obtained using conventional techniques known to those with skill in the art, and the fragments are screened for utility in the same manner as are intact antibodies.

As used herein, the term “binding” or “specifically binding” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant (epitope) and an antigen-binding site on the antibody: the lesser the value of the KD, the stronger the binding strength between an antigenic determinant (epitope) and the antibody. Alternatively, the affinity can also be expressed as the affinity constant (KA), which is 1/KD.

Avidity is the measure of the strength of binding between an antibody and the pertinent antigen. Avidity is related to both the affinity between an antigenic determinant (epitope) and its antigen binding site on the antibody and the number of pertinent binding sites present on the antibody. Typically, an antibody will bind with a dissociation constant (KD) of 10⁻⁵ to 10⁻¹² M or less, and preferably 10⁻⁷ to 10⁻¹² M or less and more preferably 10⁻⁸ to 10⁻¹² M, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ M is generally considered to indicate non-specific binding. Specifically binding of an antibody to an antigen or antigenic determinant can be determined in any suitable manner known per se, including, for example, Scatchard analysis and/or competitive binding assays, such as radioimmunoassays (RIA), enzyme immunoassays (EIA), bio-layer interferometry (BLI) assay and sandwich competition assays, and the different variants thereof known per se in the art.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

“CD117” (also referred to as c-kit or Stem Cell Factor Receptor (SCRF)) is a single transmembrane, receptor tyrosine kinase that binds the ligand Stem Cell Factor (SCF). SCF induces homodimerization of cKIT which activates its tyrosine kinase activity and signals through both the PI3-AKT and MAPK pathways. CD117 is highly expressed on hematopoietic stem cells (HSCs). This expression pattern makes CD117 a potential target for conditioning across a broad range of diseases.

As used herein, the term “CD3” refers to the human CD3 protein complex, which has five peptide chains, γ chain, δ chain, ε chain, ζ chain and η chain, and is associated with the T cell receptor a and β chains to form a TCR-CD3 complex. The term includes any CD3 variants, isoforms and species homologs which are naturally expressed by cells, including T cells, or are expressed on cells transfected with genes or cDNA encoding the aforementioned chains.

As used herein, the term “bispecific T-cell engager” or “BiTE” refers to a polypeptide chain molecule having two antigen-binding domains, one of which binds to a T-cell antigen and the second of which binds to an antigen present on the surface of target cells (See, PCT Publication WO 05/061547; Baeuerle et al., 2008, Drugs of the Future 33:137-147; Bargou, et al., 2008, Science 321:974-977, which are incorporated herein by reference in their entireties). Thus, the BiTE of the disclosure has an antigen binding region that binds to CD117 and a second antigen binding region that is directed towards a T-cell antigen.

As used herein, the term “vector” is intended to refer to a nucleic acid molecule capable of transporting another nucleic acid to which it has been linked.

As used herein, the term “host cell” refers to a cell into which an expression vector has been introduced.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and Iin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin-(negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin-(negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self renewal potential (i.e., they survive throughout adulthood, and can be serially transplanted through successive recipients), whereas ST-HSCs have limited self renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. hematopoietic disorder) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a hematopoietic disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g., hematopoietic disorders). The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment, or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc.

### Bispeicific Antibodies

The invention provides a bispeicific antibody or an antigen binding fragment thereof, comprising a first antigen binding region and a second antigen binding region, wherein the first antigen binding region binds to CD117 and comprises a first heavy chain variable region (VH_A) and a first light chain variable region (VL_A), and the second binding region binds to CD3 and comprises a second heavy chain variable region (VH_B) and a second light chain variable region (VL_B), wherein:
the VH_A comprises a HCDR1, a HCDR2 and a HCDR3 of a VH comprising the amino acid sequence of SEQ ID NO: 44;
the VL_A comprises a LCDR1, a LCDR2 and a LCDR3 of a VL comprising the amino acid sequence of SEQ ID NO: 47;
the VH_B comprises a HCDR1, a HCDR2 and a HCDR3 of a VH comprising the amino acid sequence of SEQ ID NO: 43; and
the VL_B comprises a LCDR1, a LCDR2 and a LCDR3 of a VL comprising the amino acid sequence of SEQ ID NO: 46.

In some embodiments, the CDRs are determined by Chothia definition. In some other embodiments, the CDRs are determined by Kabat definition. In some embodiments, the CDRs are determined by a composite of Chothia and Kabat definition system. In still some other embodiments, the CDRs are determined by AbM definition.

In some embodiments of the bispecific antibody or antigen binding fragment thereof,
the VH_A comprises a HCDR1, a HCDR2 and a HCDR3 having the amino acid sequences of SEQ ID NOs: 6, 12 and 18 respectively;
the VL_A comprises a LCDR1, a LCDR2 and a LCDR3 having the amino acid sequences of SEQ ID NOs: 26, 32 and 38 respectively;
the VH_B comprises a HCDR1, a HCDR2 and a HCDR3 having the amino acid sequences of SEQ ID NOs: 5, 11 and 17 respectively; and
the VL_B comprises a LCDR1, a LCDR2 and a LCDR3 having the amino acid sequences of SEQ ID NOs: 25, 31 and 37 respectively.

In some embodiments of the bispecific antibody or antigen binding fragment thereof,
the VH_A comprises an amino acid sequence having at least 80%, at least 85%, at least 90%, at least 95%, at least 98%, at least 99%, or 100% sequence identity to SEQ ID NO: 44;
the VL_A comprises an amino acid sequence having at least 80%, at least 85%, at least 90%, at least 95%, at least 98%, at least 99%, or 100% sequence identity to SEQ ID NO: 47;
the VH_B comprises an amino acid sequence having at least 80%, at least 85%, at least 90%, at least 95%, at least 98%, at least 99%, or 100% sequence identity to SEQ ID NO: 43; and
the VL_B comprises an amino acid sequence having at least 80%, at least 85%, at least 90%, at least 95%, at least 98%, at least 99%, or 100% sequence identity to SEQ ID NO: 46.

In some embodiments, the VH_A comprises a functional variant of the amino acid sequence as set forth in SEQ ID NO: 44 formed by insertion, deletion and/or substitution of one or more amino acid(s) therein, provided that the antibody comprising the VH_A comprising the functional variant retains the ability of binding to CD117. In some embodiments, the VL_A comprises a functional variant of the amino acid sequence as set forth in SEQ ID NO: 47 formed by insertion, deletion and/or substitution of one or more amino acid(s) therein, provided that the antibody comprising the VL_A comprising the functional variant retains the ability of binding to CD117. In some embodiments, the VH_B comprises a functional variant of the amino acid sequence as set forth in SEQ ID NO: 43 formed by insertion, deletion and/or substitution of one or more amino acid(s) therein, provided that the antibody comprising the VH_B comprising the functional variant retains the ability of binding to CD3. In some embodiments, the VL_B comprises a functional variant of the amino acid sequence as set forth in SEQ ID NO: 46 formed by insertion, deletion and/or substitution of one or more amino acid(s) therein, provided that the antibody comprising the VL_B comprising the functional variant retains the ability of binding to CD3.

The functional variant comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the amino acid sequence of the parent polypeptide. For example, the functional variant of SEQ ID NO: 44 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 44. For example, the functional variant of SEQ ID NO: 47 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 47. For example, the functional variant of SEQ ID NO: 43 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 43. For example, the functional variant of SEQ ID NO: 46 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 46.

In the context of the functional variant, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In a preferred embodiment, the VH_A comprises an amino acid sequence as set forth in SEQ ID NO: 44; the VL_A comprises an amino acid sequence as set forth in SEQ ID NO: 47; the VH_B comprises an amino acid sequence as set forth in SEQ ID NO: 43; and the VL_B comprises an amino acid sequence as set forth in SEQ ID NO: 46.

In some embodiments of the bispecific antibody or antigen binding fragment thereof, the antibody does not comprise an immunoglobulin Fc region.

In some other embodiments, the antibody comprises three polypeptide chains, wherein:
the first polypeptide chain comprises from the N terminal to C terminal: VH_A-CH1; the second polypeptide chain comprises from the N terminal to C terminal: VL_A-CL-L-VH_B-CH1; and the third polypeptide chain comprises from the N terminal to C terminal: VL_B-CL; or
the first polypeptide chain comprises from the N terminal to C terminal: VH_B-CH1; the second polypeptide chain comprises from the N terminal to C terminal: VL_B-CL-L-VH_A-CH1; and the third polypeptide chain comprises from the N terminal to C terminal: VL_A-CL,
wherein CH1 represents the first domain of constant region of an immunoglobulin heavy chain; CL each represents constant region of an immunoglobulin light chain; and L is absent or represent an optional linker.

In some embodiments, the CL in the second polypeptide chain and the CL in the third polypeptide chain each independently comprises a constant region derived from λ light chain or κ light chain.

In some embodiments, the CL in the second polypeptide chain and the CL in the third polypeptide chain are derived from the same type of immunoglobulin light chain (λ light chain or κ light chain). In some other embodiments, the CL in the second polypeptide chain and the CL in the third polypeptide chain are derived from different types of immunoglobulin light chains.

In case that the CL in the second polypeptide chain and the CL in the third polypeptide chain are derived from different types of immunoglobulin light chains, the bispecific antibody can be purified by using affinity chromatography according to the type of CL in the second polypeptide chain, so that the bispecific antibody comprising the second polypeptide chain associated with the first polypeptide chain and third polypeptide chain is captured while by products without the second polypeptide claim are removed.

In some embodiments, the CL in the second polypeptide chain comprises a light chain constant region derived from 2 light chain (e.g. human λ light chain), and the CL in the third polypeptide chain comprises a light chain constant region derived from κ light chain (e.g. human κ light chain).

In some other embodiments, the CL in the second polypeptide chain comprises a light chain constant region derived from κ light chain (e.g. human κ light chain), and the CL in the third polypeptide chain comprises a light chain constant region derived from λ light chain (e.g. human λ light chain).

In some embodiments of the bispecific antibody or antigen binding fragment thereof, the VL_A comprises a light chain variable region derived from the same type of immunoglobulin light chain as the CL in the same polypeptide chain, and/or the VL_B comprises a light chain variable region derived from the same type of immunoglobulin light chain as the CL in the same polypeptide chain.

In some embodiments of the bispecific antibody or antigen binding fragment thereof, each of the CH1 indepednently comprises a CH1 derived from immunoglobulin isotype IgG (e.g. human IgG), optionally each of the CH1 indepednently comprises a CH1 derived from IgG subtype selected from the group consisting of IgG1, IgG2 and IgG4 (e.g. human IgG1, IgG2 and IgG4).

In some embodiments, the linker may be any flexible linker. For example, the linker comprises an amino acid sequence of (G4S) n, wherein n is an integer selected from 1-5, e.g. 1, 2, 3, 4 or 5. In some embodiments, the linker may comprise an amino acid sequence of GGGGS. In some embodiments, the linker may comprise an amino acid sequence of GGGGSGGGGS. In some embodiments, the linker may comprise an amino acid sequence of GGGGSGGGGSGGGGS. In some embodiments, the linker may comprise an amino acid sequence of GGGGSGGGGSGGGGSGGGGS. In some embodiments, the linker may comprise an amino acid sequence of GGGGSGGGGSGGGGSGGGGSGGGGS.

In some embodiments of the bispecific antibody comprising three polypeptide chains, the first polypeptide chain comprises an amino acid sequence having at least 80%, at least 85%, at least 90%, at least 95%, at least 98%, at least 99%, or 100% sequence identity to SEQ ID NO: 57; the second polypeptide chain comprises an amino acid sequence having at least 80%, at least 85%, at least 90%, at least 95%, at least 98%, at least 99%, or 100% sequence identity to SEQ ID NO: 56; and the third polypeptide chain comprises an amino acid sequence having at least 80%, at least 85%, at least 90%, at least 95%, at least 98%, at least 99%, or 100% sequence identity to SEQ ID NO: 52.

In some embodiments, the first polypeptide chain comprises a functional variant of the amino acid sequence as set forth in SEQ ID NO: 57 formed by insertion, deletion and/or substitution of one or more amino acid(s) therein, provided that the antibody comprising the functional variant retains the ability of binding to CD117 and/or CD3. In some embodiments, the second polypeptide chain comprises a functional variant of the amino acid sequence as set forth in SEQ ID NO: 56 formed by insertion, deletion and/or substitution of one or more amino acid(s) therein, provided that the antibody comprising the functional variant retains the ability of binding to CD117 and/or CD3. In some embodiments, the third polypeptide chain comprises a functional variant of the amino acid sequence as set forth in SEQ ID NO: 52 formed by insertion, deletion and/or substitution of one or more amino acid(s) therein, provided that the antibody comprising the functional variant retains the ability of binding to CD117 and/or CD3.

For example, the functional variant of SEQ ID NO: 57 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 57. For example, the functional variant of SEQ ID NO: 56 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 56. For example, the functional variant of SEQ ID NO: 52 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 52.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-50, preferably 1-20, more preferably 1-10, still more preferably 1-5. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3 and/or FR4; and/or constant regions, e.g., CL and/or CH1.

In some embodiments, the substitution of one or more amino acid(s) can be conservative substitution of one or more amino acid(s). Examples of conservative substitutions are as described above.

In a preferred embodiment, the first polypeptide chain comprises an amino acid sequence as set forth in SEQ ID NO: 57; the second polypeptide chain comprises an amino acid sequence as set forth in SEQ ID NO: 56; and the third polypeptide chain comprises an amino acid sequence as set forth in SEQ ID NO: 52.

In some embodiments, the bispecific antibody is a bispecific T-cell engager (BiTE).

In some embodiments, the antibody or antigen binding fragment of the invention is attached to a fluorescent label, radiolabel or cytotoxic agent.

### Nucleic Acids

The invention provides a nulecic acid (e.g. an isolated nucleic acid) encoding the bispecific antibody or antigen binding fragment thereof of the invention.

The term “nucleic acid” includes both single-stranded and double-stranded nucleotide polymers. The nucleic acid can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

For example, the invention provides nucleic acid molecules encoding any one of the heavy chain variable region sequences disclosed herein. The invention also provides nucleic acid molecules that are at least 90%, at least 95%, at least 98% or at least 99% identical to nucleic acids encoding any one of the heavy chain variable region sequences disclosed herein.

For example, the invention provides nucleic acid molecules encoding any one of the light chain variable region sequences disclosed herein. The invention also provides nucleic acid molecules that are at least 90%, at least 95%, at least 98% or at least 99% identical to nucleic acids encoding any one of the light chain variable region sequences disclosed herein.

For example, the invention provides nucleic acid molecules encoding: (i) any one of the heavy chain variable region sequences disclosed herein and (ii) any one of the light chain variable region sequences disclosed herein. The invention also provides nucleic acid molecules that are at least 90%, at least 95%, at least 98% or at least 99% identical to nucleic acids encoding: (i) any one of the heavy chain variable region sequences disclosed herein and (ii) any one of the light chain variable region sequences disclosed herein.

For example, the invention provides nucleic acid molecules encoding a heavy chain variable region sequence that comprises the CDR sequences of any one of the heavy chain variable region sequences disclosed herein.

The invention also provides nucleic acid molecules that encode a heavy chain variable region sequence that comprises CDR sequences that are at least 90%, at least 95%, at least 98% or at least 99% identical to the CDR sequences of any one of the heavy chain variable region sequences disclosed herein.

For example, the invention provides nucleic acid molecules encoding a light chain variable region sequence that comprises the CDR sequences of any one of the light chain variable region sequences disclosed herein.

The invention also provides nucleic acid molecules that encode a light chain variable region sequence that comprises CDR sequences that are at least 90%, at least 95%, at least 98% or at least 99% identical to the CDR sequences of any one of the light chain variable region sequences disclosed herein.

For example, the invention provides nucleic acid molecules encoding: (i) a heavy chain variable region sequence that comprises the CDR sequences of any one of the heavy chain variable region sequences disclosed herein and (ii) a light chain variable region sequence that comprises the CDR sequences of any one of the light chain variable region sequences disclosed herein. The invention also provides nucleic acid molecules that encode: (i) a heavy chain variable region sequence that comprises CDR sequences that are at least 90%, at least 95%, at least 98% or at least 99% identical to the CDR sequences of any one of the heavy chain variable region sequences disclosed herein and (ii) a light chain variable region sequence that comprises CDR sequences that are at least 90%, at least 95%, at least 98% or at least 99% identical to the CDR sequences of any one of the light chain variable region sequences disclosed herein.

In some embodiments, the nucleic acid is ribonucleic acid (RNA) or deoxyribonucleic acid (DNA). In some embodiments, the invention provides a ribonucleic acid (RNA) comprising a nucleotide sequence encoding the bispecific antibody disclosed herein. In some embodiments, the invention provides a deoxyribonucleic acid (DNA) comprising a deoxynucleotide sequence encoding the bispecific antibody disclosed herein.

In some embodiments, the deoxyribonucleic acid (DNA) may be introduced into the cells of a human body in vivo. In some embodiments, the deoxyribonucleic acid (DNA) of the invention is comprised in a vector or a delivering agent. In some embodiments, the deoxyribonucleic acid (DNA) of the invention is integrated into the genome of a cell.

In some embodiments, the ribonucleic acid (RNA) may be introduced into the cells of a human body in vivo. In some embodiments, the ribonucleic acid (RNA) of the invention is comprised in a vector or a delivering agent.

### Vectors

The invention provides a vector comprising the nucleic acid of the invention.

In some embodiments, the vector is an expression vector capable of expressing a polypeptide comprising a heavy or light chain variable region of the bispecific antibody. For example, the invention provides expression vectors comprising any of the nucleic acid molecules mentioned above.

Any vector may be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pGAR, pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

An expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGT10, AGT11, AZapII (Stratagene), NEMBL4, and ANM1149, also may be used. Examples of plant expression vectors useful in the context of the disclosure comprise pBI01, pBI101.2, pBI101.3, pBI121 and pBIN19 (Clontech). Examples of animal expression vectors useful in the context of the disclosure comprise pcDNA, pEUK-Cl, pMAM, and pMAMneo (Clontech).

Recombinant expression vectors may be prepared using standard recombinant DNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, may be prepared to contain a replication system functional in a prokaryotic or eukaryotic host cell. Replication systems may be derived, e.g., from ColEl, 2μ plasmid, λ, SV40, bovine papilloma virus, and the like.

For example, the vector may be an adenoviral vector comprising a nucleotide sequence encoding the bispecific antibody disclosed herein. The vector may be administered into the body of a subject, and then enter into a cell of the subject in vivo, thereby the nucleotide sequence encoding the bispecific antibody disclosed herein is integrated into the genome of the cell, and subsequently the cell expresses the bispecific antibody disclosed herein.

### Host Cells

The invention also provides a host cell which comprises the nucleic acid of the invention or the vector of the invention.

Any cell may be used as a host cell for the nucleic acids or the vectors of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some embodiments, the cell is a human cell. In some embodiments, the cell is an immune cell. In some embodiments, host cells include, for example, CHO cells, such as CHOS cells and CHOK1 cells, or HEK293 cells, such as HEK293A, HEK293T and HEK293FS.

The host cell of the invention is prepared by introducing the vector disclosed herein or the nucleic acid disclosed herein in vitro or ex vivo. The host cell of the invention may be administered into the body of a subject, and the host cell expresses the bispecific antibody disclosed herein in vivo.

The invention provides host cells into which any of the vectors mentioned above have been introduced. The invention further provdes a method of preparing the bispecific antibody of the invention, wherein the method comprises a) culturing the host cell of the fourth aspect of the invention under a condition suitable for the production of the bispecific antibody; and b) obtaining the bispecific antibody from the culture.

### Antibody-Drug Conjugate

The invention also provides immunoconjugates and antibody-drug conjugates (ADCs), which comprise the bispecific antibody or antigen binding fragment thereof of the invention, and a chemical moiety or effector molecule conjugated to the bispecific antibody or antigen binding fragment thereof.

In the context of the present disclosure, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate is often referred to as an “immunoconjugate”. When the conjugate comprises an antibody linked to a drug (e.g., a cytotoxic agent), the conjugate is often referred to as an “antibody-drug conjugate” or “ADC.”

In some embodiments, the effector molecule can be a detectable label or an immunotoxin. Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain la of PE and the B chain of DT) and replacing it with a different targeting moiety, such as an antibody.

The term “conjugated” or “linked” may refer to making two polypeptides into one contiguous polypeptide molecule. In one embodiment, an antibody is joined to an effector molecule. In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule.

The invention provides immunoconjugates that include the antibody or antigen-binding fragment disclosed herein and an effector molecule. In some embodiments, the effector molecule is a toxin, such as, but not limited to, Pseudomonas exotoxin or a variant thereof. In other embodiments, the effector molecule is a detectable label, such as, but not limited to, a fluorophore, an enzyme or a radioisotope.

The disclosed antibodies can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell. Conversely, where it is desired to invoke a non-lethal biological response, the therapeutic agent can be conjugated to a non-lethal pharmacological agent or a liposome containing a non-lethal pharmacological agent.

Effector molecules can be linked to an antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site.

Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies one skilled in the art will be able to determine a suitable method for attaching a given agent to an antibody or other polypeptide.

The antibodies disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bi-specific antibody or a diabody), a detection agent, a pharmaceutical agent, and/or a protein or peptide that can mediate association of the antibody or antibody portion with another molecule (such as a strep tavidin core region or a polyhistidine tag).

One type of derivatized antibody is produced by cross-linking two or more antibodies (of the same type or of different types, such as to create bi-specific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

The bispecific antibody can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-l-napthalenesulfonyl chloride, phycoerythrin, lanthanide phosphors and the like. Bioluminescent markers are also of use, such as luciferase, green fluorescent protein (GFP) and yellow fluorescent protein (YFP).

The bispecific antibody or antigen binding fragment can also be conjugated with enzymes that are useful for detection, such as horseradish peroxidase, β-galactosidase, luciferase, alkaline phosphatase, glucose oxidase and the like. When a bispecific antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. The bispecific antibody or antigen binding fragment may also be conjugated with biotin, and detected through indirect measurement of avidin or streptavidin binding. It should be noted that the avidin itself can be conjugated with an enzyme or a fluorescent label.

The bispecific antibody may be fused to a self-labelling protein tag (e.g. HaloTag). For example, the protein tag could be cloned at the end of a constant region. HaloTag is a self-labelling protein tag derived from a bacterial enzyme (a haloalkane dehalogenase), designed to covalently bind to a synthetic ligand. In some instances, the synthetic ligand comprises a chloroalkane linker attached to a fluorophore, such as a near-infrared fluorophore (Los et al. (2008) ACS Chem Biol. 3 (6): 373-82).

The bispecific antibody may be labeled with a magnetic agent, such as gadolinium. Antibodies can also be labeled with lanthanides (such as europium and dysprosium), and manganese.

Paramagnetic particles such as superparamagnetic iron oxide are also of use as labels. The bispecific antibody may also be labeled with a predetermined polypeptide epitopes recognized by a secondary reporter (such as leucine zipper pair sequences, binding sites for secondary antibodies, metal binding domains, epitope tags). In some embodiments, labels are attached by spacer arms of various lengths to reduce potential steric hindrance.

The bispecific antibody can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

The bispecific antibody can also be derivatized with a chemical group such as polyethylene glycol (PEG), a methyl or ethyl group, or a carbohydrate group. These groups may be useful to improve the biological characteristics of the bispecific antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one embodiment, the toxin is Pseudomonas exotoxin (PE) (U.S. Pat. No. 5,602,095).

The antibodies described herein can also be used to target any number of different diagnostic or therapeutic compounds to cells expressing the antigen on their surface. Thus, a bispecific antibody of the present disclosure can be attached directly or via a linker to a drug that is to be delivered directly to cells expressing cell-surface antigen. This can be done for therapeutic, diagnostic or research purposes. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to a bispecific antibody can be an encapsulation system, such as a nanoparticle, liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well known to those of skill in the art (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Compositions

The invention also provides a composition comprising the bispecific antibody or antigen binding fragment thereof of the invention. In some embodiments, the composition is a pharmaceutical composition and further comprises a pharmaceutically acceptable carrier and/or excipient.

The bispecific antibody or antigen binding fragment thereof or agents of the invention (also referred to herein as “active compounds”), and derivatives, fragments, analogs and homologs thereof, can be incorporated into pharmaceutical compositions suitable for administration. Such compositions typically comprise the bispecific antibody or antigen binding fragment thereof or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In some embodiments of the pharmaceutical composition disclosed herein, the pharmaceutical composition further comprises a second therapeutic agent. In some embodiments, the second therapeutic agent is for depleting hematopoietic stem cells in a subject and/or treating a hematopoietic disorder in a subject.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention provides therapeutic compositions comprising the bispecific antibody or antigen binding fragment thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of bispecific antibody may vary depending upon the age and the size of a subject to be administered, target disease, conditions, route of administration, and the like. When an antibody of the present invention is used for treating a disease or disorder in an adult patient, or for preventing such a disease, it is advantageous to administer the antibody of the present invention normally at a single dose of about 0.1 to about 60 mg/kg body weight, more preferably about 5 to about 60, about 10 to about 50, or about 20 to about 50 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

### Therapeutic Methods

The invention provides a method of depleting hematopoietic stem cells in a subject in need thereof, comprising administrating the bispecific antibody of the invention or the composition of the invention to the subject. In some embodiments, a therapeutically effective amount of the bispecific antibody or the composition is administered to the subject in an amount sufficient to deplete hematopoietic stem cells in the subject.

The invention further provdes a method of treating a hematopoietic disorder in a subject in need thereof, comprising: a) administrating the bispecific antibody of the invention or the composition of the invention to the subject; and b) transplanting hematopoietic stem cells to the subject. In some embodiments, a therapeutically effective amount of the bispecific antibody or the composition is administered to the subject in an amount sufficient to deplete hematopoietic stem cells, treat a hematopoietic disorder, or inhibit a sign or a symptom of the hematopoietic disorder in the subject.

In some embodiments, the hematopoietic disorder is seleted from the gourp consisting of a hemoglobinopathy, an immunodeficiency, a metabolic disorder, a hematological disorder (such as bone marrow failure syndromes, myeloid dysplastic syndromes, acute lymphoid and myeloid leukemia, and chronic lymphoid and myeloid leukemia), a hematopoietic cell proliferative disease, transplant rejection, an autoimmune disorder and an autoinflammatory disorder.

In some embodiments, the hematopoietic disorder is selected from the group consisting of hematopoietic disorders including hemoglobinopathy, bone marrow failure syndromes, immune-deficiencies, metabolic disorders, malignancies of myeloid and lymphoid origin and hematopoietic proliferative disorders such as: mastocytosis, myeloid dysplasic syndrome (MDS), acute myeloid leukemia (AML), acute promyelocytic leukemia (APL), AMLwith recurrent genetic abnormalities (e.g. molecular changes in CD117, NPM1, CEBPalpha, FLT3, RUNX1, ASXL1, TP53, IDH1 and IDH2), chromosomal aberrations (e.g. favorable chromosomal changes including abnormalities of chromosome 16 at bands p13 and q22 [t (16; 16) (p13;q22), inv (16) (p13q22)] and a translocation between chromosomes 8 and 21 [t (8; 21)], intermediate risk aberrations including trisomy 8 and translocations with chromosome 9 and 11 [t (9; 11)] and unfavorable chromosomal changes including subgroup of cases with extra copies of chromosomes 8 or 13 [for example, trisomy 8 (+8)], deletion of all or part of chromosomes 5 or 7, complex changes on many chromosomes, and changes to chromosome 3 at band q26. AML with myelodysplasia-related changes (MDS), AML with therapy-related myeloid neoplasms, AML that is otherwise categorized of NOS (not otherwise specified), MO Myeloblastic leukemia without differentiation, M1 Myeloblastic with little of no maturation, M2 Myeloblastic with maturation, M3 Promyelocytic, M4 Myelomonocytic, M4eo Myelomonocytic with eosinophils, M5a Monocytic without differentiation (monoblastic), M5b Monocytic with differentiation, M6 Erythroleukemic, recurrent AML and relapsed AML,-c-KIT-postive acute lymphoblastic leukemia (CLL), Mast cell leukemia, Megakaryoblastic leukemia, chronic myeloid leukemia including BCR-ABL (t (9; 11) Philadelphia chromosome Ph-positive and Ph-negative CML at the chronic, accelerated and blast phase, chronic lymphoid leukemia in need for hematopoietic stem cell transplantation, such as cases with early relapse or refractory disease after purine analog based treatment or those with p53 mutations, T-lymphoblastic leukemia with CD117 receptor expression, auto-inlammatory disease such as Familial Mediterranean fever (FMF), Cryopyrin-associated periodic syndromes (CAPS), TNF-receptor-associated periodic syndrome (TRAPS), Deficiency of IL-1-receptor antagonist (DIRA), Hyper IgD syndrome (HIDS), Systemic autoinflammatory disease (SAIDS), Blau's syndrome, Mejeed syndrome, pyohenic arthritis pyoderma gangrenosum and acne syndrome (PAPA), periodic fever, aphtous stomatitis, pharyngitis and adenitis syndrome (PFAPA), Behcet's disease, Still's disease, Crohn's disease and acquired auto inflammatory syndromes like Schnitzler's syndrome.

Hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population. The bispecific antibody or the composition described herein can thus be used to treat a non-malignant hemoglobinopathy (e.g., a mastocytosis, a hemoglobinopathy selected from the group consisting of sickle cell anemia, thalassemia, Fanconi anemia, aplastic anemia, and Wiskott-Aldrich syndrome). Additionally or alternatively, the bispecific antibody or the composition described herein can be used to treat an immunodeficiency, such as a congenital immunodeficiency. Additionally or alternatively, the bispecific antibody or the composition described herein can be used to treat an acquired immunodeficiency (e.g., an acquired immunodeficiency selected from the group consisting of HIV and AIDS). The bispecific antibody or the composition described herein can be used to treat a metabolic disorder (e.g., a metabolic disorder selected from the group consisting of glycogen storage diseases, mucopolysaccharidoses, Gaucher's Disease, Hurlers sphingolipidoses, and metachromatic leukodystrophy).

Additionally or alternatively, the bispecific antibody or the composition described herein can be used to treat a malignancy or proliferative disorder, such as a hematologic cancer, myeloproliferative disease. In the case of cancer treatment, the bispecific antibody or the composition described herein may be administered to a subject so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the bispecific antibody or the composition described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the bispecific antibody or the composition described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The bispecific antibody or the composition described herein may be used to induce solid organ transplant tolerance. For instance, the bispecific antibody or the composition described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the bispecific antibody or the composition described herein may be used to induce transplant tolerance in a solid organ transplant recipient (e.g., a kidney transplant, lung transplant, liver transplant, and heart transplant, among others).

In addition, the bispecific antibody or the composition described herein can be used to treat cancers directly, such as cancers characterized by cells that are CD117+. For instance, the bispecific antibody or the composition described herein can be used to treat leukemia, particularly in subjects that exhibit CD117+ leukemic cells. By depleting CD117+ cancerous cells, such as leukemic cells, the bispecific antibody or the composition described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma.

Acute myeloid leukemia (AML) is a cancer of the myeloid line of blood cells, characterized by the rapid growth of abnormal white blood cells that build up in the bone marrow and interfere with the production of normal blood cells. AML is the most common acute leukemia affecting adults, and its incidence increases with age. The symptoms of AML are caused by replacement of normal bone marrow with leukemic cells, which causes a drop in red blood cells, platelets, and normal white blood cells. As an acute leukemia, AML progresses rapidly and may be fatal within weeks or months if left untreated. In one embodiment, the bispecific antibody or the composition described herein are used to treat AML in a human patient in need thereof. In certain embodiments the treatment with the bispecific antibody or the composition depletes AML cells in the treated subjects. In some embodiments about 50% or more of the AML cells are depleted. In other embodiments, about 60% or more of the AML cells are depleted, or about 70% or more of the AML cells are depleted, or about 80% of more or about 90% or more, or about 95% or more of the AML cells are depleted. In certain embodiments the treatment with the bispecific antibody or the composition is a single dose treatment. In certain embodiments the single dose treatment depletes about 60%, about 70%, about 80%, about 90%, about 95% or more of the AML cells.

In addition, the bispecific antibody or the composition described herein can be used to treat autoimmune disorders. For instance, the bispecific antibody or the composition can be administered to a subject, such as a human patient suffering from an autoimmune disorder, so as to kill a CD117+ immune cell. The CD117+ immune cell may be an autoreactive lymphocyte, such as a T-cell that expresses a T-cell receptor that specifically binds, and mounts an immune response against, a self antigen. By depleting self-reactive, CD117+ cells, the bispecific antibody or the composition described herein can be used to treat autoimmune pathologies, such as those described below. Additionally or alternatively, the bispecific antibody or the composition described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the bispecific antibody or the composition described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy,

Chagas' disease, chronic fatigue immune dysfunction syndrome (CFIDS), chronic inflammatory demyelinating polyneuropathy, Crohn's disease, cicatricial pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenia purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

In some embodiments of the method, the step b) is performed within 24 hours, within 48 hours, within 72 hours, within 5 days, within 7 days, within 2 weeks, within 3 weeks or within 1 month after step a).

In some embodiments, the dosage administered to a subject may vary with the embodiment, the medicament employed, the method of administration, and the site and subject being treated. However, a dose should be sufficient to provide a therapeutic response. A clinician may determine the effective amount to be administered to a human or other subject in order to treat a medical condition. The precise amount required to be therapeutically effective may depend upon numerous factors, e.g., such as the activity of the antibody, and the route of administration.

A dose of the bispecific antibodies or compositions disclosed herein may be administered to a mammal at one time or in a series of sub-doses administered over a suitable period of time, e.g., on a daily, semi-weekly, weekly, bi-weekly, semi-monthly, bi-monthly, semi-annual, or annual basis, as needed. A dosage unit comprising an effective amount of the bispecific antibodies or the compositions may be administered in a single daily dose, or the total daily dosage may be administered in two, three, four, or more divided doses administered daily, as needed.

A suitable means of administration may be selected by a medical practitioner. Route of administration may be parenteral, for example, administration by injection, transnasal administration, transpulmonary administration, or transcutaneous administration. Administration may be systemic or local by intravenous injection, intramuscular injection, intraperitoneal injection, subcutaneous injection. In some embodiments, the bispecific antibodies or the compositions are selected for parenteral delivery, for inhalation, or for delivery through the digestive tract, such as orally. Dose and method of administration may vary depending on the weight, age, condition, and the like of the subject, and may be suitably selected.

In some embodiments, the method further comprises administering to the subject a second therapeutic agent.

In certain embodiments, the bispecific antibody or the composition disclosed herein is administered prior to, substantially simultaneously with, or after the administration of the second therapeutic agent.

In some embodiments, the second therapeutic agent is selected from an antibody, a chemotherapeutic agent and a small molecule drug. In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, or glucocorticoid. In some embodiments, the second therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

### Kits

The invention provides a pharmaceutical pack or kit comprising one or more containers filled with one or more of the ingredients of the compositions described herein, such as the bispecific antibodies disclosed herein. Optionally, associated with such container(s) can be a notice in the form prescribed by a governmental agency regulating the manufacture, use or sale of pharmaceuticals or biological products, which notice reflects approval by the agency of manufacture, use or sale for human administration.

In a specific embodiment, the kit comprises a first container containing the bispecific antibodies disclosed herein. In a specific embodiment, the kit comprises a first container that is a vial containing the bispecific antibodies as a lyophilized sterile powder under vacuum, and the kit further comprises a second container comprising a pharmaceutically acceptable fluid.

In a specific embodiment, provided herein is an injection device containing the bispecific antibodies. In a specific embodiment, the injection device comprises the bispecific antibody in sterile solution. In a specific embodiment, the injection device is a syringe.

### Medical Uses

The invention provides use of the bispecific antibody or the composition disclosed herein in the manufacture of a medicament for depleting hematopoietic stem cells in a subject.

The invention provides use of the bispecific antibody or the composition disclosed herein in the manufacture of a medicament for treating a hematopoietic disorder in a subject.

The invention also provides the bispecific antibody or the composition disclosed herein for use in depleting hematopoietic stem cells in a subject.

The invention further provides the bispecific antibody or the composition disclosed herein for use in treating a hematopoietic disorder in a subject.

In some embodiments of the use disclosed herein, the bispecific antibody or the composition disclosed herein is in combination with a second therapeutic agent. In some embodiments, the second therapeutic agent is selected from an antibody, a chemotherapeutic agent and a small molecule drug. In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, or glucocorticoid. In some embodiments, the second therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some embodiments, the hematopoietic disorder is seleted from the gourp consisting of a hemoglobinopathy, an immunodeficiency, a metabolic disorder, a hematological disorder (such as bone marrow failure syndromes, myeloid dysplastic syndromes, acute lymphoid and myeloid leukemia, and chronic lymphoid and myeloid leukemia), a hematopoietic cell proliferative disease, transplant rejection, an autoimmune disorder and an autoinflammatory disorder.

In some embodiments, the hematopoietic disorder is selected from the group consisting of hematopoietic disorders including hemoglobinopathy, bone marrow failure syndromes, immune-deficiencies, metabolic disorders, malignancies of myeloid and lymphoid origin and hematopoietic proliferative disorders such as: myeloid dysplasic syndrome (MDS), acute myeloid leukemia (AML), acute promyelocytic leukemia (APL), AMLwith recurrent genetic abnormalities (e.g. molecular changes in CD117, NPM1, CEBPalpha, FLT3, RUNX1, ASXL1, TP53, IDH1 and IDH2), chromosomal aberrations (e.g. favorable chromosomal changes including abnormalities of chromosome 16 at bands p13 and q22 [t (16; 16) (p13; q22), inv (16) (p13q22)] and a translocation between chromosomes 8 and 21 [t (8; 21)], intermediate risk aberrations including trisomy 8 and translocations with chromosome 9 and 11 [t (9; 11)] and unfavorable chromosomal changes including subgroup of cases with extra copies of chromosomes 8 or 13 [for example, trisomy 8 (+8)], deletion of all or part of chromosomes 5 or 7, complex changes on many chromosomes, and changes to chromosome 3 at band q26. AML with myelodysplasia-related changes (MDS), AML with therapy-related myeloid neoplasms, AML that is otherwise categorized of NOS (not otherwise specified), MO Myeloblastic leukemia without differentiation, M1 Myeloblastic with little of no maturation, M2 Myeloblastic with maturation, M3 Promyelocytic, M4 Myelomonocytic, M4eo Myelomonocytic with eosinophils, M5a Monocytic without differentiation (monoblastic), M5b Monocytic with differentiation, M6 Erythroleukemic, recurrent AML and relapsed AML,-c-KIT-postive acute lymphoblastic leukemia (CLL), Mast cell leukemia, Megakaryoblastic leukemia, chronic myeloid leukemia including BCR-ABL (t (9; 11) Philadelphia chromosome Ph-positive and Ph-negative CML at the chronic, accelerated and blast phase, chronic lymphoid leukemia in need for hematopoietic stem cell transplantation, such as cases with early relapse or refractory disease after purine analog based treatment or those with p53 mutations, T-lymphoblastic leukemia with CD117 receptor expression, auto-inlammatory disease such as Familial Mediterranean fever (FMF), Cryopyrin-associated periodic syndromes (CAPS), TNF-receptor-associated periodic syndrome (TRAPS), Deficiency of IL-1-receptor antagonist (DIRA), Hyper IgD syndrome (HIDS), Systemic autoinflammatory disease (SAIDS), Blau's syndrome, Mejeed syndrome, pyohenic arthritis pyoderma gangrenosum and acne syndrome (PAPA), periodic fever, aphtous stomatitis, pharyngitis and adenitis syndrome (PFAPA), Behcet's disease, Still's disease, Crohn's disease and acquired auto inflammatory syndromes like Schnitzler's syndrome.

Autoimmune diseases that can be treated using the bispecific antibody or the composition described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatricial pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopeniarpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

### Methods of Production

The antibodies of the invention are produced by any technique known in the art, such as, without limitation, any chemical, biological, genetic or enzymatic technique, either alone or in combination.

Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

### EXAMPLES

### Antibody Sequences

The antibody sequences of PR004384 anti-CD117xCD3 Bispecific T cell engager, PR001310 anti-HELxCD3 control Bispecific T cell engager, and the recombinant monoclonal antibody PR004382 are shown in following Tables 1-3.

TABLE 1
Antibody sequences of PR004384

| | | SEQ ID |
|---|---|---|
| PR004384 | Sequence | NO: |
| Chain 1 | DIVMTQSPDSLAVSLGERATINCRASESVDIYGNSFMHWYQQKPGQPPKLL | 57 |
| | IYLASNLESGVPDRFSGSGSGTDFTLTISSLQAEDVAVYYCQQNNEDPYTFG | |
| | GGTKVEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGECEVQLVESGGGLVQPGGSLKLSCAASGFTFNTYAMNW | |
| | VRQASGKGLEWVGRIRSKYNNYATYYADSVKDRFTISRDDSKSTLYLQMN | |
| | SLKTEDTAVYYCVRHGNFGNSYVSWFAYWGQGTLVTVSSASTKGPSVFPL | |
| | APSSKSTSGGTAALGCLVKDYFPEPVTVSWNSGALTSGVHTFPAVLQSSGL | |
| | YSLSSVVTVPSSSLGTQTYICNVNHKPSNTKVDKKVEPKSC | |
| Chain 2 | QVQLVQSGAEVKKPGASVKVSCKASGYTFTSYNMHWVRQAPGQGLEWM | 56 |
| | GVIYSGNGDTSYNQKFKGRVTITADKSTSTAYMELSSLRSEDTAVYYCARE | |
| | RDTRFGNWGQGTLVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNV | |
| | NHKPSNTKVDKKVEPKSC | |
| Chain 3 | QAVVTQEPSLTVSPGGTVTLTCRSSTGAVTTSNYANWVQQKPGQAPRGLI | 52 |
| | GGTNKRAPWTPARFSGSLLGDKAALTLLGAQPEDEAEYFCALWYSNLWV | |
| | FGGGTKLTVLGQPKAAPSVTLFPPSSEELQANKATLVCLISDFYPGAVTVA | |
| | WKADSSPVKAGVETTTPSKQSNNKYAASSYLSLTPEQWKSHRSYSCQVTH | |
| | EGSTVEKTVAPTECS | |
| VH for | QVQLVQSGAEVKKPGASVKVSCKASGYTFTSYNMHWVRQAPGQGLEWM | 44 |
| CD117 | GVIYSGNGDTSYNQKFKGRVTITADKSTSTAYMELSSLRSEDTAVYYCARE | (SEQ ID |
| binding | RDTRFGNWGQGTLVTVSS | NOs: 6, 12 |
| (CDRs in | | and 18 for |
| Bold) | | the CDRs) |
| VL for | DIVMTQSPDSLAVSLGERATINCRASESVDIYGNSFMHWYQQKPGQPPKL | 47 |
| CD117 | LIYLASNLESGVPDRFSGSGSGTDFTLTISSLQAEDVAVYYCQQNNEDPYT | (SEQ ID |
| binding | FGGGTKVEIK | NOs: 26 |
| (CDRs in | | 32 and 38 |
| Bold) | | for the |
| | | CDRs) |
| VH for CD3 | EVQLVESGGGLVQPGGSLKLSCAASGFTFNTYAMNWVRQASGKGLEWV | 43 |
| binding | GRIRSKYNNYATYYADSVKDRFTISRDDSKSTLYLQMNSLKTEDTAVYYC | (SEQ ID |
| (CDRs in | VRHGNFGNSYVSWFAYWGQGTLVTVSS | NOs: 5, 11 |
| Bold) | | and 17 for |
| | | the CDRs) |
| VL for CD3 | QAVVTQEPSLTVSPGGTVTLTCRSSTGAVTTSNYANWVQQKPGQAPRGLI | 46 |
| binding | GGTNKRAPWTPARFSGSLLGDKAALTLLGAQPEDEAEYFCALWYSNLWV | (SEQ ID |
| (CDRs in | FGGGTKLTVL | NO: 25, 31 |
| Bold) | | and 37 for |
| | | the CDRs) |

TABLE 2
Antibody sequences of PR001310

| | | SEQ ID |
|---|---|---|
| PR001310 | Sequence | NO: |
| Chain 1 | DIVLTQSPAIMSASPGEKVTMTCSASSSVNYMYWYQQKSGTSPKRWIYDTS | 55 |
| | KLASGVPVRFSGSGSGTSYSLTISSMETEDAAEYYCQQWGRNPTFGGGTKL | |
| | EIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVT | |
| | KSFNRGECEVQLVESGGGLVQPGGSLKLSCAASGFTFNTYAMNWVRQASG | |
| | KGLEWVGRIRSKYNNYATYYADSVKDRFTISRDDSKSTLYLQMNSLKTED | |
| | TAVYYCVRHGNFGNSYVSWFAYWGQGTLVTVSSASTKGPSVFPLAPSSKS | |
| | TSGGTAALGCLVKDYFPEPVTVSWNSGALTSGVHTFPAVLQSSGLYSLSSV | |
| | VTVPSSSLGTQTYICNVNHKPSNTKVDKKVEPKSC | |
| Chain 2 | EVQLQQSGAELMKPGASVKISCKASGYTFSDYWIEWVKQRPGHGLEWIGE | 54 |
| | ILPGSGSTNYHERFKGKATFTADTSSSTAYMQLNSLTSEDSGVYYCLHGNY | |
| | DFDGWGQGTTLTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNH | |
| | KPSNTKVDKKVEPKSC | |
| Chain 3 | QAVVTQEPSLTVSPGGTVTLTCRSSTGAVTTSNYANWVQQKPGQAPRGLI | 52 |
| | GGTNKRAPWTPARFSGSLLGDKAALTLLGAQPEDEAEYFCALWYSNLWV | |
| | FGGGTKLTVLGQPKAAPSVTLFPPSSEELQANKATLVCLISDFYPGAVTVA | |
| | WKADSSPVKAGVETTTPSKQSNNKYAASSYLSLTPEQWKSHRSYSCQVTH | |
| | EGSTVEKTVAPTECS | |

TABLE 3
sequences of PR004382

| | | SEQ ID |
|---|---|---|
| PR004382 | Sequence | NO: |
| Heavy chain | QVQLVQSGAEVKKPGASVKVSCKASGYTFTSYNMHWVRQAPGQGLEWM | 50 |
| | GVIYSGNGDTSYNQKFKGRVTITADKSTSTAYMELSSLRSEDTAVYYCARE | |
| | RDTRFGNWGQGTLVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNV | |
| | NHKPSNTKVDKKVEPKSCDKTHTCPPCPAPELLGGPDVFLFPPKPKDTLMIS | |
| | RTPEVTCVVVDVSHEDPEVKFNWYVDGVEVHNAKTKPREEQYNSTYRVV | |
| | SVLTVLHQDWLNGKEYKCKVSNKALPAPEEKTISKAKGQPREPQVYTLPPS | |
| | REEMTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| Light chain | DIVMTQSPDSLAVSLGERATINCRASESVDIYGNSFMHWYQQKPGQPPKLL | 53 |
| | IYLASNLESGVPDRFSGSGSGTDFTLTISSLQAEDVAVYYCQQNNEDPYTFG | |
| | GGTKVEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |

### Cell Culture

Human erythroleukemic TF-1 cells were grown in complete medium A (RPMI 1640 medium supplemented with 10% fetal bovine serum (FBS; Gibco), L-Glutamine at 2 mM (Life Technologies), penicillin at 100 U/mL (Gibco) and Streptomycin-sulfate at 100 μg/mL (Gibco), Sodium Pyruvate at 1 mM (Life Technologies) and human granulocyte-macrophage colony-stimulating factor (GM-CSF) at 5 ng/ml (Peprotech)). TF-1 cells were maintained at a concentration of 2-9×10⁵ cells/mL in a humidified incubator with CO₂ (5%) at 37° C.

Primary human CD34+ from bone marrow cells were purchased from STEMCELL Technologies. Cells were seeded at 5×10⁴ cells/mL and expanded in serum-free StemSpan SFEM II media supplemented with StemSpan CD34+ Expansion Supplement, UM729 (1 μM) and SR-1 (0.5 μM) (STEMCELLS Technologies, Vancouver, BC, Canada) for a maximum 7 days. Cells were diluted to a concentration of 1×10⁵ cells/mL after 3 or 4 days. For human bone marrow-CD34+ assay, complete medium B (serum-free StemSpan SFEM II media supplemented with stem cell factor (SCF) at 100 ng/mL, thrombopoietin (TPO) at 100 ng/mL, Flt3 ligand at 100 ng/mL (Peprotech®, NJ, USA), UM729 at 1 μM and SR-1 at 0.5 μM) was used.

Human multiple myeloma RPMI-8226 and acute myeloid leukemia U937 cells were expanded in RPMI-1640 medium supplemented with FBS (10%), L-Glutamine (2 mM), penicillin (100 U/mL), at 37° C. in a humidified incubator with CO₂ (5%).

Human PBMCs were isolated from healthy donors (Sanquin, Netherlands) by Ficoll density centrifugation (GE Healthcare, Illinois, USA). Human CD3-positive T lymphocytes were purified from frozen human PBMCs using RosetteSep™ Human T Cell Enrichment Cocktail (STEMCELLS Technologies) according to the manufacturer's instructions. Purified T cells were plated in a 24-wells plate at a concentration of 1×10⁶ cells/mL in complete medium C (RPMI 1640 medium supplemented with FBS 10%, L-Glutamine at 2 mM, penicillin at 100 U/mL and Streptomycin-sulfate at 100 μg/mL with human Interleukin-2 (hIL-2) at 20UI/mL (PeproTech)) overnight. For isolation of human NK cells, RosetteSep™ Human NK Cell Enrichment Cocktail (STEMCELLS Technologies) was used according to the manufacturer's instructions. All the samples were tested for purity (>90%) on the BD LSRII-Fortessa (BD Biosciences) and using FlowJo VX (BD Biosciences) for data analysis. Subsequently, NK cell density was adjusted at 1×10⁶ cells/mL in 24-wells plate in complete medium C.

### Flow Cytometry-Based Binding Assay for Bispecific T Cell Engager

TF-1 cells, human PBMCs or purified human CD3-positive T cells (2-5×10⁵ cells) were washed in FACS wash buffer (FWB), resuspended in 50 μL of Bispecific T cell engager or antibody solutions at indicated concentrations in FWB and incubated on ice for 2 hours. For negative control, human CD3-positive T cells were pre-incubated with mouse anti-human CD3 (BD) (10 μg/mL) at 4° C. for 30 minutes. Cells were washed twice and incubated with Alexa Fluor 647 goat anti-human-F (ab′) 2 fragment for the detection of the Bispecific T cell engagers (Jackson ImmunoResearch Laboratories, PA, USA) at room temperature (RT) for 1 hour, followed by two washes in FWB and fixation in paraformaldehyde (1%), CaCl₂) (1 mM) solution. RPMI-8226 and U937 cell lines, both cell lines that do not express CD117, were used as negative controls. Expression of CD117 was determined with a BD LSRII-Fortessa (BD Biosciences) and data were analyzed with FlowJo (LLC, CA, USA) software.

### Flow Cytometry-Based Binding Assay for PR004382

Recombinant monoclonal antibody PR004382 was prepared into FACS buffer (PBS with 2% FBS) by 5-fold serial dilution. TF-1 cells were washed in FWB, resuspended into 96-well V-bottom plates at 1×10⁵ cells per well with 100 μL of diluted antibody solutions, and incubated at 4° C. for 60 minutes. Cells were washed twice and incubated with secondary antibody (Alexa Fluor® 647 AffiniPure F(ab′)₂ Fragment Goat Anti-Human IgG, Fcγ fragment specific, 1:1000 dilution, Jason ImmunoResearch, cat #109-606-008) at 4° C. for 60 minutes, followed by two washes in FWB and fixation. Fluorescence was read on ACEA NovaCyte Flow Cytometer, and data were analyzed with FlowJo (LLC, CA, USA) software, and then processed and plotted by GraphPad Prism.

### ELISA-Based Binding Assay for PR004382

CD117 protein (SinoBiological, cat #11996-H08H) was coated onto 96-well plates at 1 μg/ml, 100 μl/well at 4° C. overnight. The plates were washed for 3 times with 200 μl/well wash buffer (0.05% BSA, 1X PBS, 0.05% Tween); and then incubated with 200 μl/well blocking buffer (2% BSA, 1X PBS, 0.05% Tween) for 2 hours at room temperature. Testing antibodies were prepared into wash buffer by 5-fold serial dilution. The diluted antibodies were added into plates at 100 μl/well, and then incubated for 1 hour at room temperature. The plates were washed for 3 times and incubated with secondary antibody (Anti-Human IgG (Fc specific)-Peroxidase antibody produced in goat, Sigma, cat #A0170-1ML) for 30 minutes at room temperature. After washing for 3 times, 100 μl/well of TMB was added into plates, and then incubated at room temperature for 15 minutes, before ELISA stop solution was added. Absorption at 450 nm was recorded by Plate Reader (Molecular Devices, SpectraMax). The data were processed and plotted by GraphPad Prism.

### Flow Cytometry Stainings

In case of whole blood samples (20-50 μL) from mice, red blood cells were lysed in 2 mL of NH₄Cl (150 mM), KHCO₃ (10 mM), 0.1 mM EDTA (ACK lysis buffer) on ice for 2 minutes. For staining of surface antigens on hematopoietic cell types, cells were washed with FWB and incubated with a 20 μL mix of monoclonal antibodies (Table 4: Monoclonal antibodies) at 4° C. for 20 min. After staining, cells were washed in FWB and fixed with PFA (1%) CaCl₂) (1 mM) on ice. In order to exclude apoptotic and dead cells from the analysis, Annexin-V antibody (BD Biosciences) and CaCl₂) (2.5 mM) were added to the staining solutions. Signals were determined on a BD LSRII-Fortessa flow cytometer system (BD Biosciences), with FACS Diva software. Data were analyzed with FlowJo VX (BD Biosciences).

TABLE 4
Monoclonal antibodies

| | Catalog | |
|---|---|---|
| Reagents | Number | Company |
| Alexa Fluor ® 700 anti-mouse | 103127 | BioLegend Europe BV |
| CD45 antibody | | |
| PerCp-eF710 anti-murine CD3 | 46-0037-42 | eBioscienc |
| antibody | | |
| BV421 anti-human CD117 | 313216 | BioLegend Europe BV |
| FITC anti-human CD45 antibody | 304006 | BioLegend Europe BV |
| APC anti-human CD34 antibody | 343607 | BioLegend Europe BV |
| BV711 anti-human CD69 antibody | 563836 | BioLegend Europe BV |
| PE-CF594 anti-human CD3 | 562280 | BioLegend Europe BV |
| antibody | | |
| BV650 anti-human CD4 antibody | 563875 | BioLegend Europe BV |
| BV786 anti-human CD8 antibody | 563823 | BioLegend Europe BV |
| SB600 anti-human CD25 antibody | 63-0259-42 | eBioscienc |
| PerCP-Cy ™5.5 Annexin V | 561431 | BD Pharmingen |

### T Cell-Dependent Cellular Cytotoxicity (TDCC) Assay

Target cells (TF-1 or human bone marrow-CD34+ cells) were labeled with CellTrace™ FarRed/CFSE fluorescent dye (ThermoFisher Scientific) according to the manufacturer's protocol. Labeled cells (1×10⁶ cells/mL, 50 μL/well) were seeded in a 96-well round-bottom plate together with 100 μL of human CD3-positive T cells at indicated effector-to-target (E:T) ratios in complete medium A or B (see above). 50 μL of complete media containing Bispecific T cell engagers at indicated concentrations was added to each well and incubated at 37° C., 5% CO₂ for 6 or 24 hours. After two washes, the cells were stained with Annexin-V. Cells were tested by flow cytometry and at least 10,000 total events within the target cell gate were collected. To measure the specific target cell lysis, CellTrace™-labeled target cells were gated and examined for Annexin-V positivity. The percentage of specific lysis was then calculated using the following equation: Cytotoxicity (%)= [Dead target cell (%)−spontaneous death (%)]*100/[100−spontaneous death (%)].

### Cytokine Analysis

To examine the cytokine production of T cells in response to Bispecific T cell engager treatment in vitro, supernatants were collected from the TDCC assay and centrifuged to remove residual debris or intact cells. All samples were stored at −20° C. for a maximum of one month. After thawing samples on ice, they were centrifuged at 1,300×g for 5 minutes at room temperature. Two-fold dilutions were prepared in low-protein-binding plates (Corning™ 96-Well Nonbinding Surface [NBS™] Microplates) according to the manufacturer's instructions (Ella automated Immunoassay system, Biotechne). Subsequently, the samples were diluted at 1:1 in wash buffer according to manufacturer's instructions for supernatant. Diluted samples were loaded into ELLA Simple Plex cartridges and analyzed with the ELLA Simple Plex system (Biotechne).

### T Cell Activation Assay

To measure the T cell activation, cells from TDCC assays were harvested after 24 hours. Cells were stained with fluorescent antibodies anti-CD4-BV650, anti-CD8-BV786, anti-CD69-BV711 (Biolegend Europe BV) and anti-CD25-SB600 (eBioscience). CellTrace™-labeled target cells were excluded from the analysis. Annexin-V negative CD4+ and CD8+ T cells were analyzed for CD25 and CD69 expression with FlowJo VX (BD Biosciences).

### T Cell Proliferation Assay

To analyze T cell proliferation, 0.5×10⁵ TF-1 target cells were incubated with FarRed-labelled purified T cells (E: T=10:1) in complete medium A (see above) containing indicated Bispecific T cell engagers at indicated concentrations and 37° C. for 5 days. Cells were harvested and stained with Annexin-V. Data were acquired by running the samples on the BD LSRII-Fortessa (BD Biosciences). Proliferation of T cells (Annexin-V⁻/CellTrace™⁺) was analyzed with FlowJo VX (BD Biosciences).

### Cross-Activity Test Bispecific T Cell Engagers on Murine T Cells

Murine CD3-positive T cells were purified from spleens of C57BL6/J mice by negative selection with magnetic beads (EasySep, Stemcell Technologies) and cultured in complete medium C overnight. To measure the cross-activity of Bispecific T cell engagers, TF-1 target cells were labelled with CellTrace™ according to the manufacturer's protocol. Then, labelled TF-1 target cells (1×10⁶ cells/mL, 50 μL) were plated in complete medium A in a 96-well round-bottom plate and 100 μl CD3-positive T cells (E: T=4:1) were added. Bispecific T cell engagers (100 ng/ml) in complete medium A were added to each well and plates were incubated in an incubator at 37° C., 5% CO₂ for 24 hours. To analyze the murine T cell-induced specific target cell lysis, the cells were harvested and stained with Annexin-V and anti-murine-CD3-PerCp-eF710. The TF-1 cells (CellTrace™-positive cells) were examined using flow cytometry and the same gate strategy used for the TDCC assay.

### Pharmacokinetics (pK) of Bispecific T Cell Engagers

Female C57BL6/J mice (8-10 week old) (Charles River France) were injected intraperitoneally (IP) with 1.0 mg/kg of anti-CD117xCD3 Bispecific T cell engager diluted in sterile saline (0.9%). Blood samples (20-50 μL) were collected from the tail vein into Microvette CB300 Capillary Blood Collection Tube with clotting activator (Sarstedt) at indicated time-points post-injection. After serum isolation by centrifugation at 10,000 g at RT for 5 min, the serum samples were aliquot and stored at −20° C. The anti-CD117xCD3 Bispecific T cell engager concentrations in serum were measured by enzyme-linked immunosorbent assay (ELISA) within 4 weeks post-isolation. For ELISA assay, 100 μL human recombinant CD117 protein isoform 2 (P10721-2), extracellular domain (Met 1-Thr 516) and with a poly histidine (His) tag at the C-terminus (Bio-Connect BV) (2 μg/mL in coating buffer [carbonate/bicarbonate buffer]) was used to coat the multi-array 96-well sector plates at 4° C. overnight. Plates were washed three times with 200 μl of PBST (PBS+Tween-20 [0.05%]), and blocked with PBST BSA (5%) at room temperature for 2 h. After thawing on ice, samples were centrifuged at 13,000×g at RT for 5 min. Samples were diluted (1:100-1:2000) in dilution buffer (PBST BSA [1%]) and 50 μL was added per well and incubated at 4° C. overnight. For Bispecific T cell engager detection and after washing with PBS-T, goat anti-Human IgG, 100 μL F(ab′) 2 fragment specific and HRP conjugated antibody (Jackson ImmunoResearch Laboratories, PA, USA) (1:10,000 in blocking buffer) was added at RT for 1 h in shaker. After washing, 50 μL 3,3′,5,5′-tetramethylbenzidine solution (TMB) solution was added to the wells for at least 15 minutes. Next, 50 μL H₂SO₄, 2N (stopping solution) was added. The signal was detected with a Versamax microplate reader (Molecular Devices) at 450/650 nm. The samples for calibration curve with concentrations ranging from 20 to 0.039 ng/mL CD117 protein were prepared by spiking in a pooled blank serum samples from untreated mice.

### Pharmacokinetics of PR004382

B6.Cg-Fcgrttm1Dcr Tg (CAG-FCGRT) 276Dcr/DcrJ mice (8-to-10-week-old) from Jackson Laboratory were injected intraperitoneally (IP) with PR004382 (1 mg/kg diluted in sterile 0.9% saline). Blood samples (20-50 μL) were taken from the tail vein at different time-points (1, 2, 3, 5, 7 days post-injection) with Microvette CB300 Capillary Blood Collection Tube with clotting activator (Sarstedt). Serum samples were obtained by centrifugation of the blood at 10,000 g for 5 minutes at RT, aliquoted and stored at −20° C. The analysis of the samples by ELISA was performed with the same protocol as described for the Bispecific T cell engager, except that a Goat anti-human IgG, Fcγ fragment specific, HRP conjugated antibody (Jackson ImmunoResearch Laboratories, West Grove, PA) was used.

### Anti-CD117xCD3 Bispecific T Cell Engager Treatment of Humanized NSG Mice

Female mice (16 to 24-weeks old) transplanted with human CD34+ hematopoietic stem cells (hu-CD34 NSG (005557), were purchased from Jackson Laboratory. Blood samples for Peripheral blood analysis were collected from tail vein one day before treatment. Human blood cells (hCD45+ cells) in peripheral blood were determined by flow cytometry with FITC-labeled anti-hCD45 antibodies as described below. Mice containing >70% human CD45+ cells in PB were used. Bispecific T cell engagers were diluted in sterile saline solution (0.9%) to a final concentration of 1.0 mg/kg, and injected IP every 12 hours and in total 6 times. Sixteen hours and 4.5 days post-treatment, mice were sacrificed by cervical dislocation and bone marrow cells from femur and tibiae and peripheral blood cells were collected. Cells were stained with flow cytometry surface staining protocol (see above). Hematopoietic stem and progenitor cells in the bone marrow were determined by staining the cells with an antibody cocktail containing: FITC-hCD45, AF680-mCD45, APC-hCD34, BV421-CD117 (all Biolegend Europe BV) and PerCp 5.5-Annexin-V (BD Pharmingen). Data was acquired with the BD LSRII-Fortessa (BD Biosciences).

### Antibody-Dependent Cell-Cytotoxicity Assay (ADCC) Assay

TF-1 cells and CD16A-expressing Jurkat-NFAT reporter cells were suspended into buffer (RMI1640+4% FBS) at density of 6×10⁵/ml and 2×10⁶/ml respectively. And then 50 μL of TF-1 cells (3×10⁴) and 50 μL of Jurkat-CD16a-NFAT cells (1×10⁵) were added into plates (ViewPlate™ 96 TC, PerkinElmer, cat #6005181). Testing antibodies were prepared into buffer by 3-fold serial dilution, and then 50 μL diluted antibodies were added into the plates. Plates were incubated in 5% CO₂ incubator at 37° C. for 6 hours. 50 μL/well Bright-Glo Luciferase Assay reagent was added into plates before reading luminescence on Envision plate reader (Perkinelmer). The data were processed and plotted by GraphPad Prism.

### Octet Biosensor Affinity Determination of PR004382 to the Cd16a Receptor

The testing antibodies were diluted to 200 nM in PBS buffer. HISIK sensors (Pall ForteBio, cat #18-5120) were inserted into sensor tray of Octet Red 96e (Pall ForteBio) and prepared in PBS buffer for 10 minutes. Human CD16a (V176) protein (NovoProtein, cat #C441) was loaded onto HIS1K sensors to reach loading signal level >0.2 nm, and then the senors were dipped into the wells containing the testing antibodies. The binding signals were recorded and analyzed with 1:1 global fitting model by ForteBio Data Analysis Software.

### NK-Mediated Cytotoxicity Assay

TF-1 and human bone marrow-CD34+were labeled with CellTrace™ FarRed/CFSE fluorescent dye (ThermoFisher Scientific) according to the manufacturer's protocol. Target cells were co-cultured with human NK cells at an E:T ratio of 1:1 in complete medium A (TF-1 cells) or complete medium B (human bone marrow-CD34+ cells) in an U-bottom 96 wells plate. PR004382 and IgGk1 isotype control antibody (human IgGk1, kappa-Isotype Control Antibody CBMAB-0402ZL, purchased from Creative Biolabs) were diluted in complete medium A or complete medium B and added to the cells (100 μL/well) with a final concentration ranging from 1 ng/mL to 1 μg/mL. The cells were harvested after 24 hours and stained for Annexin V. Target cells were gated on the CellTrace™ positivity and then examined for cell death by up-take of Annexin-V. At least 10,000 events within the gate for living target cells were collected. The cytotoxicity was calculated using the following equation: Cytotoxicity (%)= [Dead target cell (%)−spontaneous death (%)]×100/[100−spontaneous death (%)].

### Statistics

All statistical analyses were performed with Graphpad Prism software version 9 (GraphPad, La Jolla, CA). Four-parameter variable slope nonlinear regression was used for dose response curve fitting and for PK analysis. Data are presented as means±SEM, and compared with the two-tailed independent samples Student's t test, unless otherwise specified in the Section of Description of the Drawings. Each in vitro experiment was performed at least in triplicate. The number of asterisks indicate the level of significance of p-adjusted values: *p<0.05, **p<0.01, ***p<0.001, and ****p<0.0001.

### Ethics Statement

Animal work was performed under project licence nr. 2010607 provided by the animal welfare body (AWB) of the Instantie voor Dierenwelzijn (IvD). All animal experiments were conducted in compliance with the Netherlands' government laws of the Centrale Commissie Dierproeven (CCD). All the mice were housed in a certified barrier facility at Erasmus University Medical Center.

### Example 1. Binding Specificity of PR004384 (Anti-CD117xCD3 Bispecific T Cell Engager)

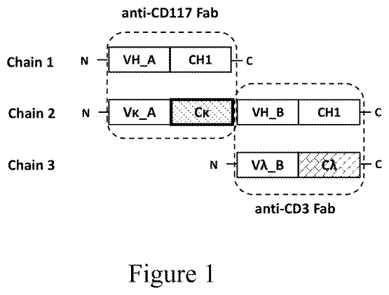

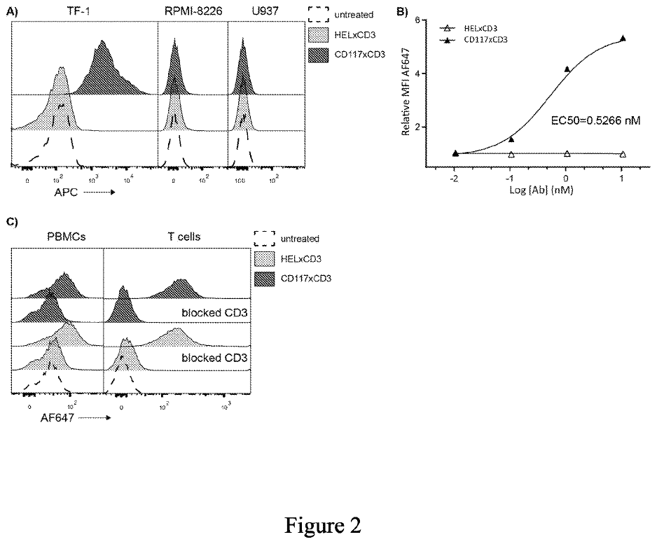

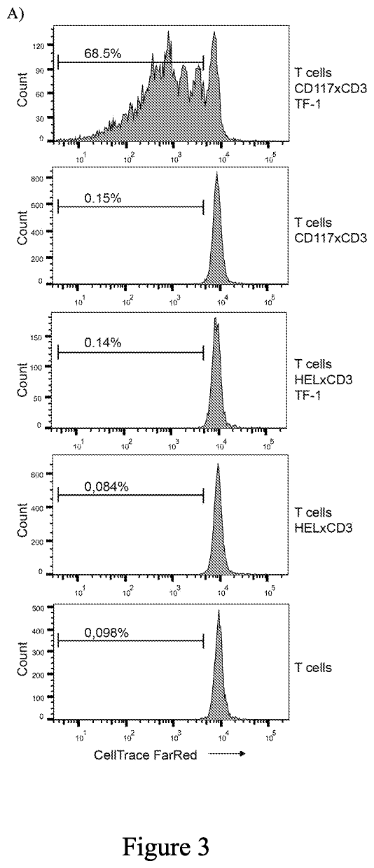

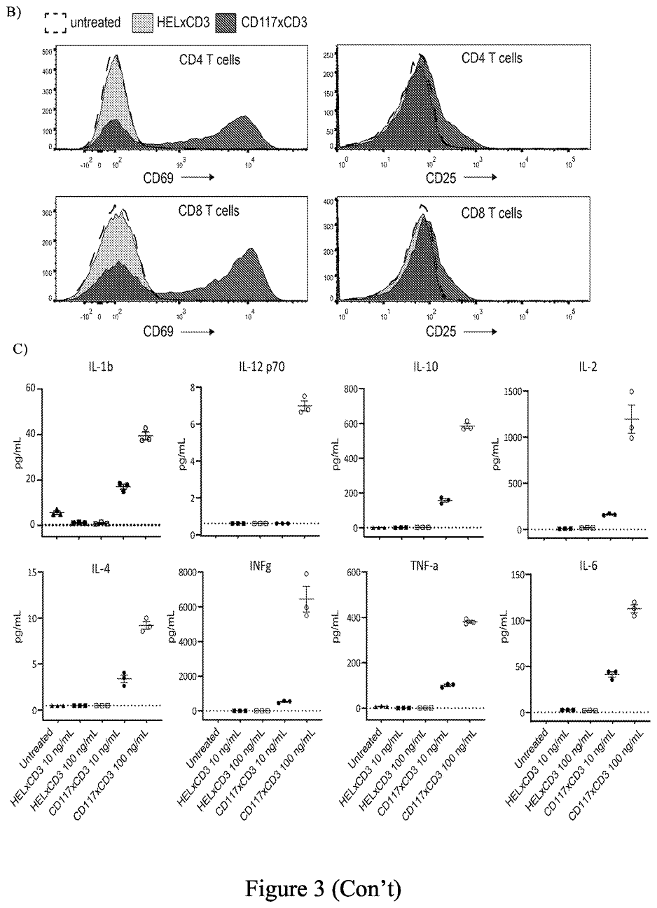

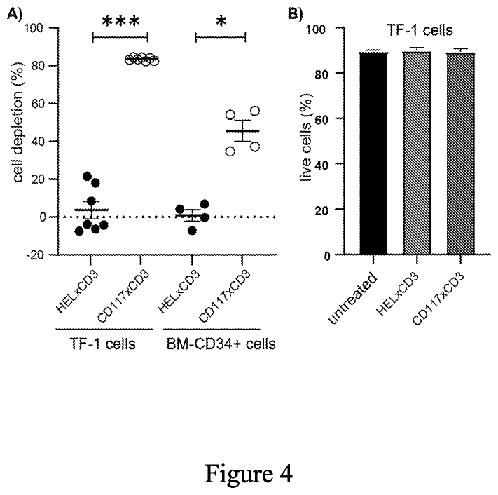

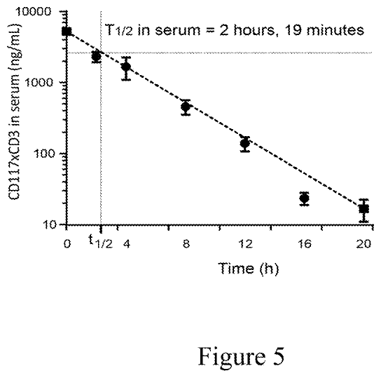

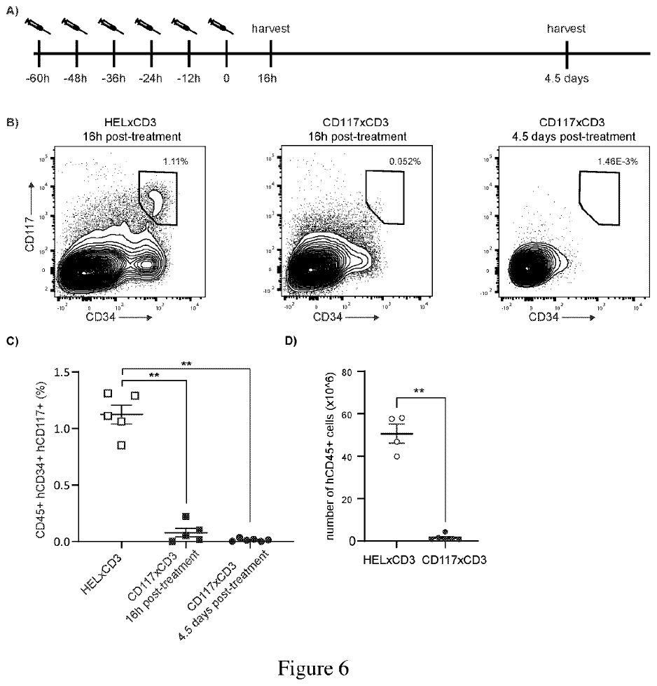

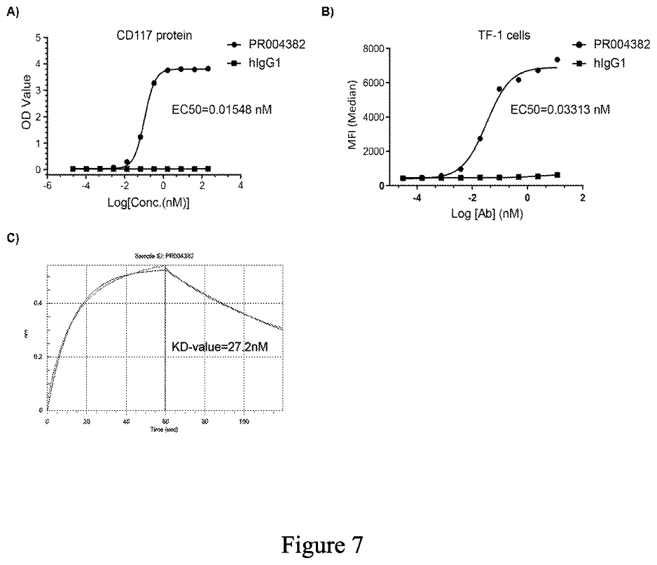

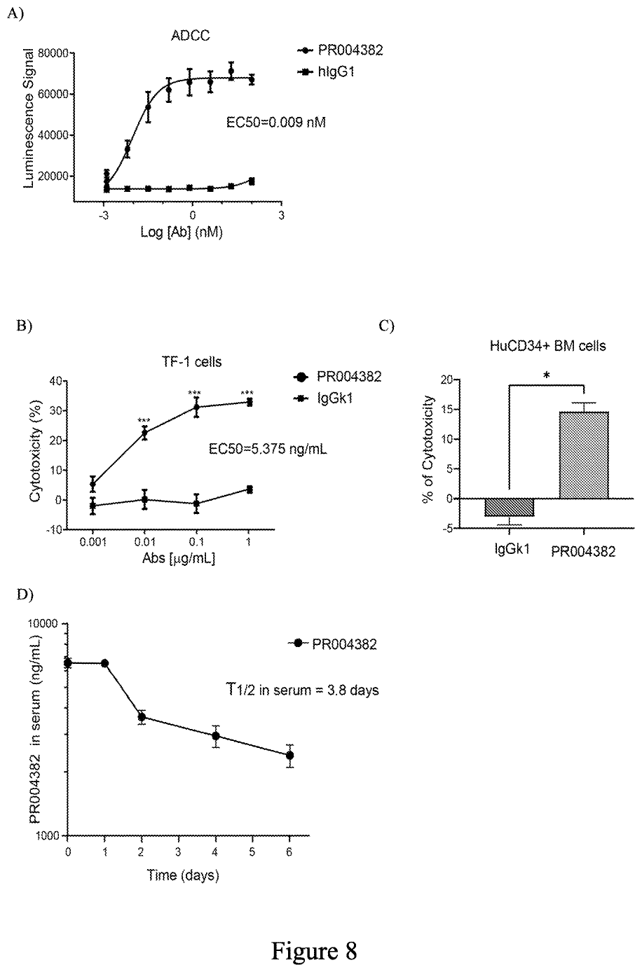

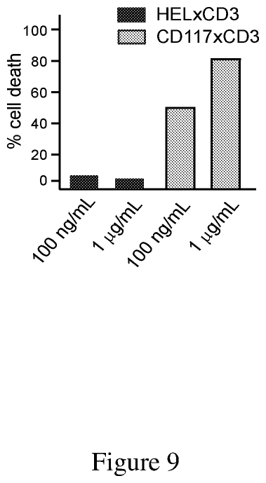

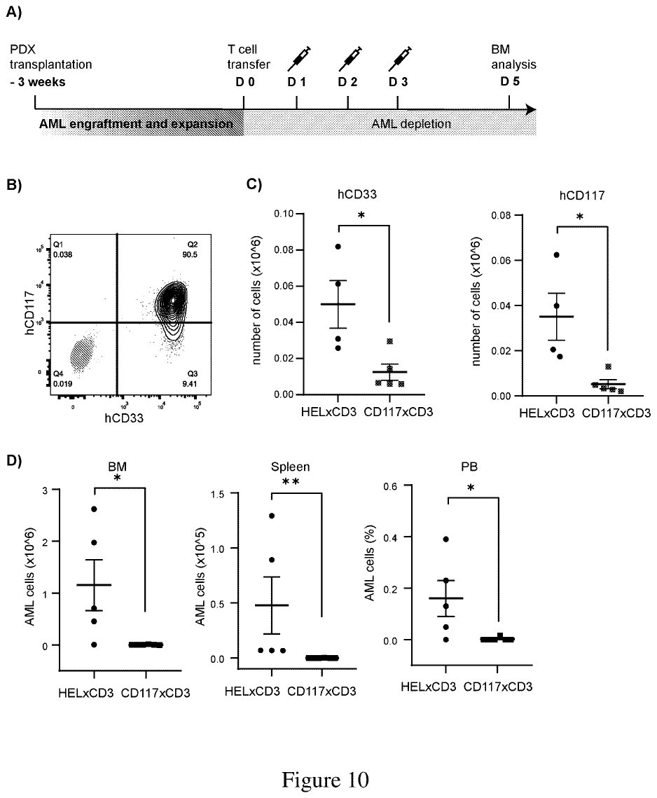

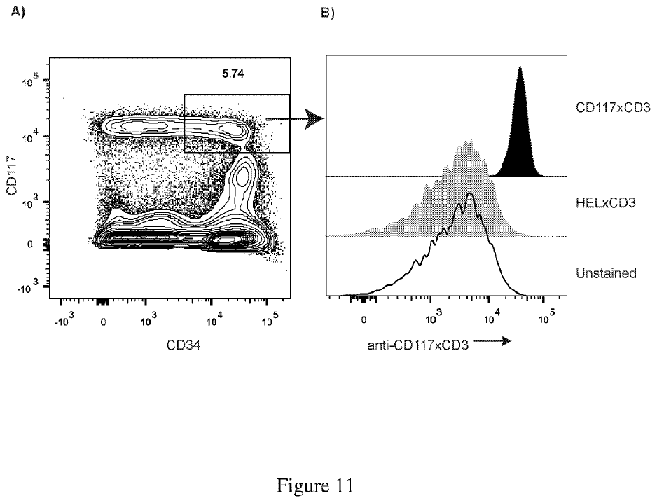

FIG. 1 shows the schematic structure of PR004384 anti-CD117xCD3 Bispecific T cell engager. In order to investigate the activities of PR004384 anti-CD117xCD3 Bispecific T cell engager, in vitro binding assays were performed with human erythroid leukemia cell line TF-1, which is known to express the CD117 receptor, and cell lines RPMI-8226 and U937, two hematopoietic cell lines that do not express CD117, as negative controls. The results show that PR004384 anti-CD117xCD3 Bispecific T cell engager binds to TF-1 cells, but not to RPMI-8226 and U937 cells (FIG. 2A). No binding of PR001310 anti-HELxCD3 control Bispecific T cell engager (HEL: hen egg lysozyme) to TF1 cells was observed (FIG. 2A). In addition, the results demonstrate that PR004384 anti-CD117xCD3 Bispecific T cell engager binds to TF-1 cells in a dose-dependent manner, and with a half maximal effective concentration (EC50) of 0.5266 nM (FIG. 2B). These results confirmed the specificity of PR004384 for CD117 receptor.

To determine the binding capacity of the PR004384 anti-CD117xCD3 Bispecific T cell engager to CD3 expressed on T cells, in vitro binding assays were performed with purified human CD3-positive T cells or total peripheral blood mononuclear cells (PBMCs). The results show that both PR004384 anti-CD117xCD3 and PR001310 anti-HELxCD3 bind specifically to purified CD3-positive T cells and CD3-positive T cells in PBMCs (FIG. 2C). Notably, no binding of both Bispecific T cell engagers could be detected when the CD3 antigens on T cells were blocked with anti-CD3 monoclonal antibody (Purified NA/LE Mouse Anti-Human CD3 Clone UCHT1 ([Catalog No: 555329, Invitrogen]) that targets the same epitope (FIG. 2C). In addition, no binding was detected for either PR004384 anti-CD117xCD3 or PR001310 anti-HELxCD3 to cells without CD3 expression such as U937 and RPMI-8226 cells (FIG. 2A), again confirming the CD3 specificity of the Bispecific T cell engagers.

Next it was tested that whether PR004384 anti-CD117xCD3 Bispecific T cell engager can simultaneously bind to TF-1 cells and T cells and activate T cells. Under in vitro cell culture conditions, T cells did not proliferate in the absence of TF-1 cells and Bispecific T cell engagers (FIG. 3A). In contrast, it was found that the PR004384 anti-CD117xCD3 Bispecific T cell engager, but not PR001310 anti-HELxCD3 Bispecific T cell engager, induced proliferation of T cells in the presence of TF-1 cells (FIG. 3A). These results showed that the T cells cultured with TF-1 cells and PR004384 anti-CD117xCD3 Bispecific T cell engager were activated by PR004384 anti-CD117xCD3 Bispecific T cell engager by simultaneously binding to CD117 and CD3 receptors.

Consistently, it was noted that the PR004384 anti-CD117xCD3 Bispecific T cell engager, but not PR001310 anti-HELxCD3 Bispecific T cell engager, induces the expression of CD69 and CD25 activation markers on T cells (FIG. 3B), as well as the expression of a panel of cytokines that are associated with T cell activation (FIG. 3C). In addition, PR004384 anti-CD117xCD3 Bispecific T cell engager upregulated Th1 and Th2 cytokines in a dose-dependent manner in the presence of both TF-1 cells and T cells (FIG. 3C).

Together, these results demonstrate that PR004384 anti-CD117xCD3 Bispecific T cell engager binds specifically and simultaneously to CD117-expressing target cells and CD3-positive T cells and activates the T cells.

### Example 2. Cytotoxic Activity of PR004384

Next, it was investigated that whether PR004384 anti-CD117xCD3 Bispecific T cell engager has hematopoietic stem cell-specific biocidal activity in vitro. In this regard, the killing of both the CD117-positive TF-1 cells and primary human bone marrow CD34+hematopoetic stem cells was tested by incubating these cells with PR004384 anti-CD117xCD3 Bispecific T cell engager or the anti-HELxCD3 as control in the presence of T cells. PR004384 anti-CD117xCD3 Bispecific T cell engager induced a more than 80% depletion of TF-1 cells after 6 hours and a more than 50% depletion of primary human bone marrow CD34+ cells after 24 hours in vitro (FIG. 4A). No depletion of CD117-positive cells was observed when the cells were treated with PR001310 anti-HELxCD3 control Bispecific T cell engager (FIG. 4A). Moreover, the anti-CD117xCD3 Bispecific T cell engager has no cross-reactivity with mouse T cells as determined by the lack of TF-1 cell killing capacity in an in vitro killing assay (FIG. 4B).

### Example 3. In Vivo Pharmacokinetics (PK) of PR004384

Then the in vivo PK of the PR004384 anti-CD117xCD3 Bispecific T cell engager was studied. In brief, PR004384 anti-CD117xCD3 Bispecific T cell engager (1.0 mg/kg) was injected into female C57BL6/J mice intraperitoneally (IP) and blood samples from the tail vein at different time points were collected for analysis. The concentration of PR004384 anti-CD117xCD3 Bispecific T cell engager in serum was determined by ELISA. The results show that the PR004384 anti-CD117xCD3 Bispecific T cell engager has a serum half-life (T½-value) of 2 hours and 19 minutes (FIG. 5).

### Example 4. In Vivo Depletion of Hematopoietic Stem Cells by PR004384

In this example, the hematopoietic stem cell depleting activity of PR004384 anti-CD117xCD3 Bispecific T cell engager in vivo was studied. The experiments were performed with humanized mice containing >70% human CD45-positive cells in the peripheral blood (PB), which were injected with PR004384 anti-CD117xCD3 Bispecific T cell engager every 12 hours for six times (FIG. 6A). No Bispecific T cell engagers in the blood was detected at 24 hours after the last injection. Notably, up to 95% depletion of human CD34+ CD117+ hematopoietic stem cells in the bone marrow of PR004384 anti-CD117xCD3 Bispecific T cell engager treated humanized mice at 16 hours after the last treatment (FIG. 6B) and an almost full depletion of CD34+ CD117+ cells at 4.5 days after the last treatment (FIG. 6C) were observed. No depletion of CD34+ CD117+ cells was detected when mice were treated with anti-HELxCD3 Bispecific T cell engager (FIG. 6C). Six weeks post-treatment, very low number of hCD45+ cells (<1.5×10⁶ cells, around 3%) were observed in the bone marrow of CD117xCD3-treated mice, whereas around 50×10⁶ hCD54+ cells on average were observed in the bone marrow of control HELxCD3-treated mice (FIG. 6D)

### Example 5, CD117 and CD16a Binding of PR004382 Monoclonal Antibody

Then the binding properties of monoclonal antibody PR004832, which has the same VH/VL sequences as the CD117 binding region of PR004384 anti-CD117xCD3 Bispecific T cell engager, were tested. In brief, binding assays were performed with PR004382 monoclonal antibody and an isotype control human IgG1 monoclonal antibody (CBMAB-0402ZL, purchased from Creative Biolabs) in an ELISA assay with CD117 protein as the target. The results show that PR004382 binds to CD117 with an EC50 of 0.01548 nM (FIG. 7A). In addition, flow cytometry-based binding assays were conducted with TF-1 cells. In this assay, an EC50 of 0.03313 nM for PR004382 was observed, whereas the hIgG1 did not bind to TF-1 cells (FIG. 7B).

To determine the affinity of PR004382 to CD16a receptor expressed on NK cells, biosensor binding assays were performed and the results show that PR004382 binds to CD16a with a KD-value of 27.2 nM (association constant K-on=3.43E+5 M, dissociation constant K-off-9.33E-03M) (FIG. 7C).

### Example 6. Cytotoxic Potential of PR004382

To investigate the cytotoxic capacity of PR004382, antibody-dependent cytotoxicity (ADCC) reporter bioassays were performed. We determined an ADCC EC50 of 0.009 nM, whereas no signals were observed for the hIgG1 control antibody CBMAB-0402ZL (FIG. 8A). Next, we performed in vitro cytotoxicity assays to determine the killing capacity of PR004382. With TF-1 cells, we determined an EC50 of 5.375 ng/ml and no cytotoxicity was determined for the IgGk1 control (FIG. 8B). Using HuCD34+ bone marrow cells as target, we determined an in vitro PR004382-mediated cytotoxicity of 15%, whereas no cytotoxicity was observed with the IgGk1 control (FIG. 8C). In addition, we determined the in vivo PK of the PR004382. For this analysis, we injected female B6.Cg-Fcgrttm1Dcr Tg (CAG-FCGRT) 276Dcr/DcrJ mice with PR004382 and collected blood samples from the tail vein at different time points for analysis. We established a serum T½-value of PR004382 of 3.8 days (FIG. 8D).

### Example 7: PR004384 Depletes Primary Human Leukamia In Vitro

In this example, depletion of primary c-KIT-positive acute leukemia cells (T/myeloid mixed phenotype acute leukemia) by PR004384 anti-CD117xCD3 Bispecific T cell engager was studied. In specific, purified leukemia cells from a patient (1×10⁵ cells) were mixed with normal human T cells (effector: target ration=10:1) in expansion medium (IMDM medium, FBS (20%), SCF, TPO, FLT3 (all 100 ng/mL), P/S) and treated with PR004384 anti-CD117xCD3 Bispecific T cell engager or anti-HELxCD3 control and incubated for 24 hours. Around 50% of cell death was observed with PR004384 at 1 μg/mL, and around 80% cell death was observed with PR004384 at 100 μg/mL, whereas no depletion of AML cells was observed when cells were treated with anti-HELxCD3 Bispecific T cell engager (FIG. 9).

### Example 8: PR004384 Depletes Human AML Cells In Vivo

In this example, capability of AML depletion by PR004384 anti-CD117xCD3 Bispecific T cell engager in humanized mice transplanted with patient-derived xenograft (PDX) AML cells (normal karyotype, FLT3-internal tandem duplication [ITD]) was studied. The mice were generated using NOD.Cg-Prkdescid Il2rgtm1Wjl Tg (CMV IL3, CSF2, KITLG) 1Eav/MloySzJ (NSG-SGM3) mice (The Jackson Laboratory, Bar Harbour, ME, USA). Briefly, AML PDX cells (1×10⁶ cells) were injected intravenously (i.v.) into sub-lethally irradiated 7-9-week-old female NSG-SGM3 mice and expanded for 3 weeks. Human purified T cells were activated for 48 hours using the T Cell TransAct kit (Miltenyi Biotec), according to the manufacturer's instructions. Three weeks post-transplantation, 1×10⁷ pre-activated human T-cells were injected intraperitoneally (i.p.) (day 0), followed by two i.p injections of 1.0 mg/kg anti-CD117xCD3 Bispecific T cell engager or anti-HELxCD3 control reagent for 3 days (2 injections per day). The presence of PDX AML cells and human T-cells were assessed by flow cytometry at day 5. Processed cells from bone marrow were analyzed by flow cytometry using anti-mCD45 FITC, anti-hCD45 APC-Cy7, anti-hCD3 PE-CF594, anti-hCD33 APC, anti-hCD4 BV650, anti-hCD8 BV785, anti-CD117 PE-CY7 (all from Biolegend) and Hoechst for viability (Invitrogen).

Humanized mice were transplanted with PDX AML cells expressing hCD117 and hCD33 (as described above), at −3 weeks of the scheme (FIG. 10A, B). After PDX AML expansion, mice were injected with activated human T cells and treated with PR004384 or control anti-HELxCD3 (FIG. 10A). A significant depletion of human PDX AML cells was observed in the bone marrow of PR004384 treated mice compared to control treated mice (FIG. 10C).

Next, depletion of leukemia initiating cells was studied in the PR004384 treated mice. In specific, anti-hCD45-Biotin and Streptavidin MicroBeads UltraPure from Miltenyi were used to enrich human cells from bone marrow of PR004384 and CTRL-treated mice. In addition, the EasySep™ Human CD3 Positive Selection Kit II (STEMCELL Technology) was used to deplete residual human T-cells. Approximately 0.2×10⁶ isolated bone marrow cells were transplanted into sub-lethally irradiated 4-6-week-old female NSG-SGM3 mice. The presence of PDX AML cells in the blood, bone marrow and spleen of secondary transplanted mice was assessed by flow cytometry 6 months post-transplantation.

Most mice that received the control treated bone marrow cells showed PDX AML cells in the bone marrow (BM), spleen and peripheral blood (FIG. 10D). However, PDX AML cells was not detectable in mice transplanted with bone marrow cells of the PR004384-treated mice (FIG. 10D), indicating that leukemia initiating cells were fully depleted by PR004384 treatment.

### Example 9: PR004384 Anti-CD117xCD3 Bispecific T Cell Engager Binds to Rhesus Macaque CD117-Positive Hematopoietic Stem Cells

In this example, whether PR004384 Bispecific T cell engager binds to CD117-positive hematopoietic stem cells of Rhesus Macaque was studied. In specific, CD34+ cells were enriched from whole bone marrow sample of Rhesus Macaque, using EasySep APC Positive Deletion Kit (STEMCELL Technology) and anti-CD34 (Clone 563, BD Pharmingen™) APC Mouse Anti-Human, according to the manufacturer's protocols. Then, the CD34-enriched cells (1×10⁶ cells, 55% purity) were washed in FACS wash buffer, resuspended in 50 μL of Bispecific T cell engagers (10 μg/mL) or FACS wash buffer only and incubated on ice for 2 hours. Cells were washed twice and incubated with R-Phycoerythrin-AffiniPure F(ab′) 2 Fragment Goat Anti-Human IgG, F(ab′) 2 Fragment for detection of the Bispecific T cell engagers (Jackson ImmunoResearch Laboratories, PA, USA) on ice for 1 hour, followed by two washes in FACS wash buffer and fixation in paraformaldehyde (1%), CaCl₂) (1 mM) solution.

Expression of CD34 and CD117 was determined with a BD LSRII-Fortessa (BD Biosciences) and data were analyzed with FlowJo (LLC, CA, USA) software (FIG. 11A). The results show that PR004384 anti-CD117xCD3 binds to CD117+ hematopoietic stem cells of Rhesus macaque (FIG. 11B).

1. A bispecific antibody or an antigen binding fragment thereof, comprising a first antigen binding region and a second antigen binding region, wherein the first antigen binding region binds to CD117 and comprises a first heavy chain variable region (VH_A) and a first light chain variable region (VL_A), and the second binding region binds to CD3 and comprises a second heavy chain variable region (VH_B) and a second light chain variable region (VL_B), wherein:
the VH_A comprises a HCDR1, a HCDR2 and a HCDR3 of a VH comprising the amino acid sequence of SEQ ID NO: 44;
the VL_A comprises a LCDR1, a LCDR2 and a LCDR3 of a VL comprising the amino acid sequence of SEQ ID NO: 47;
the VH_B comprises a HCDR1, a HCDR2 and a HCDR3 of a VH comprising the amino acid sequence of SEQ ID NO: 43; and
the VL_B comprises a LCDR1, a LCDR2 and a LCDR3 of a VL comprising the amino acid sequence of SEQ ID NO: 46.

2. The bispecific antibody or antigen binding fragment thereof according to claim 1, wherein:
the VH_A comprises a HCDR1, a HCDR2 and a HCDR3 having the amino acid sequences of SEQ ID NOs: 6, 12 and 18 respectively;
the VL_A comprises a LCDR1, a LCDR2 and a LCDR3 having the amino acid sequences of SEQ ID NOs: 26, 32 and 38 respectively;
the VH_B comprises a HCDR1, a HCDR2 and a HCDR3 having the amino acid sequences of SEQ ID NOs: 5, 11 and 17 respectively; and
the VL_B comprises a LCDR1, a LCDR2 and a LCDR3 having the amino acid sequences of SEQ ID NOs: 25, 31 and 37 respectively.

3. The bispecific antibody or antigen binding fragment thereof according to claim 1, wherein:
the VH_A comprises an amino acid sequence having at least 80%, at least 85%, at least 90%, at least 95%, at least 98%, at least 99%, or 100% sequence identity to SEQ ID NO: 44;
the VL_A comprises an amino acid sequence having at least 80%, at least 85%, at least 90%, at least 95%, at least 98%, at least 99%, or 100% sequence identity to SEQ ID NO: 47;
the VH_B comprises an amino acid sequence having at least 80%, at least 85%, at least 90%, at least 95%, at least 98%, at least 99%, or 100% sequence identity to SEQ ID NO: 43; and
the VL_B comprises an amino acid sequence having at least 80%, at least 85%, at least 90%, at least 95%, at least 98%, at least 99%, or 100% sequence identity to SEQ ID NO: 46.

4. (canceled)

5. The bispecific antibody or antigen binding fragment thereof according to claim 1, wherein the antibody comprises three polypeptide chains, wherein:
the first polypeptide chain comprises from the N terminal to C terminal: VH_A-CH1; the second polypeptide chain comprises from the N terminal to C terminal: VL_A-CL-L-VH_B-CH1; and the third polypeptide chain comprises from the N terminal to C terminal: VL_B-CL; or
the first polypeptide chain comprises from the N terminal to C terminal: VH_B-CH1; the second polypeptide chain comprises from the N terminal to C terminal: VL_B-CL-L-VH_A-CH1; and the third polypeptide chain comprises from the N terminal to C terminal: VL_A-CL,
wherein CH1 represents the first domain of constant region of an immunoglobulin heavy chain; CL each represents constant region of an immunoglobulin light chain; and L is absent or represent an optional linker.

6. The bispecific antibody or antigen binding fragment thereof according to claim 5, wherein the CL in the second polypeptide chain and the CL in the third polypeptide chain each independently comprises a constant region derived from λ light chain or κ light chain.

7. The bispecific antibody or antigen binding fragment thereof according to claim 5, wherein the CL in the second polypeptide chain and the CL in the third polypeptide chain are derived from different types of immunoglobulin light chains.

8. The bispecific antibody or antigen binding fragment thereof according to claim 7, wherein:
the CL in the second polypeptide chain comprises a light chain constant region derived from λ light chain (e.g. human λ light chain), and the CL in the third polypeptide chain comprises a light chain constant region derived from κ light chain; or
the CL in the second polypeptide chain comprises a light chain constant region derived from κ light chain, and the CL in the third polypeptide chain comprises a light chain constant region derived from λ light chain.

9. The bispecific antibody or antigen binding fragment thereof according to claim 5, wherein the VL_A comprises a light chain variable region derived from the same type of immunoglobulin light chain as the CL in the same polypeptide chain, and/or the VL_B comprises a light chain variable region derived from the same type of immunoglobulin light chain as the CL in the same polypeptide chain.

10. The bispecific antibody or antigen binding fragment thereof according to claim 5, wherein each of the CH1 independently comprises a CH1 derived from immunoglobulin isotype IgG, optionally each of the CH1 independently comprises a CH1 derived from IgG subtype selected from the group consisting of IgG1, IgG2 and IgG4.

11. The bispecific antibody or antigen binding fragment thereof according to claim 5, wherein the linker comprises an amino acid sequence of (G4S) n, wherein n is an integer selected from 1-5.

12. The bispecific antibody or antigen binding fragment thereof according to claim 5, wherein:
the first polypeptide chain comprises an amino acid sequence having at least 80%, at least 85%, at least 90%, at least 95%, at least 98%, at least 99%, or 100% sequence identity to SEQ ID NO: 57;
the second polypeptide chain comprises an amino acid sequence having at least 80%, at least 85%, at least 90%, at least 95%, at least 98%, at least 99%, or 100% sequence identity to SEQ ID NO: 56; and
the third polypeptide chain comprises an amino acid sequence having at least 80%, at least 85%, at least 90%, at least 95%, at least 98%, at least 99%, or 100% sequence identity to SEQ ID NO: 52.

13. The bispecific antibody or antigen binding fragment thereof according to claim 5, wherein:
the first polypeptide chain comprises an amino acid sequence as set forth in SEQ ID NO: 57;
the second polypeptide chain comprises an amino acid sequence as set forth in SEQ ID NO: 56; and
the third polypeptide chain comprises an amino acid sequence as set forth in SEQ ID NO: 52.

14. The bispecific antibody or antigen binding fragment thereof according to claim 1, wherein the antibody does not comprise an immunoglobulin Fc region.

15. A nucleic acid encoding the bispecific antibody or antigen binding fragment thereof according to claim 1.

16-18. (canceled)

19. A antibody drug conjugate (ADC) comprising the bispecific antibody or antigen binding fragment thereof according to claim 1, and a cytotoxic moiety conjugated to the bispecific antibody or antigen binding fragment thereof.

20. A composition comprising:
(1) the bispecific antibody or antigen binding fragment thereof according to claim 1 or an ADC comprising the bispecific antibody or the antigen binding fragment thereof according to claim 1 and a cytotoxic moiety conjugated to the bispecific antibody or antigen binding fragment thereof; or
(2) the bispecific antibody or the antigen binding fragment thereof according to claim 1 or the ADC, and a pharmaceutically acceptable carrier and/or excipient.

21. (canceled)

22. A method of depleting hematopoietic stem cells in a subject in need thereof, comprising administrating to the subject the bispecific antibody or antigen binding fragment thereof according to claim 1, an ADC comprising the bispecific antibody or antigen binding fragment thereof according to claim 1 and a cytotoxic moiety conjugated to the bispecific antibody or antigen binding fragment thereof, or a composition comprising the bispecific antibody or antigen binding fragment thereof according to claim 1.

23. A method of treating a hematopoietic disorder in a subject in need thereof, comprising:
a) administrating to the subject the bispecific antibody or antigen binding fragment thereof according to claim 1, an ADC comprising the bispecific antibody or antigen binding fragment thereof accordingly to claim 1 and a cytotoxic moiety conjugated to the bispecific antibody or antigen binding fragment thereof, or a composition comprising the bispecific antibody or antigen binding fragment thereof according to claim 1; and
b) transplanting hematopoietic stem cells to the subject.

24. The method of claim 23, wherein the step b) is performed within 24 hours, within 48 hours, within 72 hours, within 5 days, within 7 days, within 2 weeks, within 3 weeks or within 1 month after step a).

25. The method of claim 23, wherein the hematopoietic disorder is selected from the group consisting of a hemoglobinopathy, an immunodeficiency, a metabolic disorder, a hematological disorder (such as bone marrow failure syndromes, myeloid dysplastic syndromes, acute lymphoid and myeloid leukemia, and chronic lymphoid and myeloid leukemia), a hematopoietic cell proliferative disease, transplant rejection, an autoimmune disorder and an autoinflammatory disorder.
